(19) United States
(12) Patent Application Publication
(10) Pub. No.: US 2002/0111274 A1
(43) Pub. Date: Aug. 15, 2002

(54) 1-alkyl-3-[1-(substituted phenyl) benzotriazol-6-yl] uracils

(75) Inventors: Robert E. Diehl, Yardley, PA (US); Susan Trotto, Yardley, PA (US); Michael Anthony Guaciaro, East Windsor, NJ (US); Peter John Wepplo, Princeton, NJ (US)

Correspondence Address: BASF Corporation, Intellectual Property Department, P.O. Box 400, Princeton, NJ, 08543-0400, US

(73) Assignee: BASF Aktiengesellschaft, Ludwigshafen (DE)

(21) Appl. No.: 09/902,875

(22) Filed: Jul. 11, 2001

Related U.S. Application Data
(60) Provisional application No. 60/218,511, filed on Jul. 15, 2000.

(51) Int. Cl. A01N 43/54; C 07D 4 3/02

(52) U.S. Cl. 504/243; 544/310

(57) ABSTRACT

There is provided a uracil substituted 1-phenylbenzotriazole compound having the structural formula I.

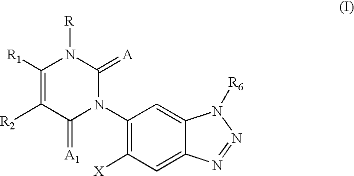

Also provided are a composition and a method comprising that compound for the control of undesirable plant species. Further provided is an intermediate compound useful in the preparation of the compound of formula I.

### BACKGROUND OF THE INVENTION

Weeds cause tremendous global economic losses by reducing crop yields and lowering crop quality. Worldwide, agronomic crops must compete with hundreds of weed species.

In spite of the commercial herbicides available today, damage to crops caused by weeds still occurs. Accordingly, there is ongoing research to create more effective and/or more selective herbicidal agents.

Certain 1-phenylbenzotriazole herbicidal agents are described in U.S. Pat. No. 5,523,277. This patent generically discloses benzotriazoles which are substituted with a Q group which may be one of 22 specified groups. However, only one Q group (Q₆) is exemplified in this patent.

Certain uracil substituted benzotriazole herbicidal agents are described in U.S. Pat. No. 5,661,108. This patent discloses benzotriazoles substituted in the 1-position by hydrogen, straight or branched chain alkyl, alkenyl, hydrozyalkyl, alkoxyalkyl, haloalkyl, alkoxycarbonyl, or arylalkyl. Substitution in the 1-position with (substituted) phenyl is however not disclosed.

Surprisingly, it has now been found the certain of the compounds generically disclosed in U.S. Pat. No. 5,523,277 are more effective and/or more selective herbicidal compounds wherein Q is Q₆.

It is therefore an object of the present invention to provide compounds which are highly effective for the control of undesirable plant species.

It is also an object of the present invention to provide methods for the selective control of undesirable plant species in the presence of crops.

Those and other objects and features of the present invention will become more apparent from the detailed description thereof set forth below.

### SUMMARY OF THE INVENTION

The present invention describes uracil-substituted 1-phenylbenzotriazole compounds which are useful as herbicidal agents.

The uracil-substituted 1-phenylbenzotriazole compounds of the present invention have the structural formula I.

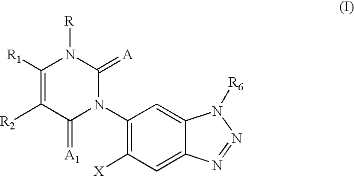

wherein

R is C₁-C₆alkyl, C₁-C₆haloalkyl, C₃-C₇cycloalkyl, C₃-C₆ alkenyl, C₃-C₆alkynyl, benzyl, OR₃ or NR₄R₅;

R₁ and R₂ are each independently hydrogen, halogen, C₁-C₆ alkyl, C₁-C₆haloalkyl, C₃-C₇cycloalkyl, C₃-C₆alkenyl or C₁-C₆alkynyl;

R₃, R₄ and R₅ are each independently hydrogen, C₁-C₆alkyl, C₁-C₆haloalkyl, C₂-C₆alkoxyalkyl, C₃-C₇cycloalkyl, C₃-C₆alkenyl, C₃-C₆alkynyl or benzyl;

X is hydrogen or halogen;

A and A₁ are each independently O or S;

R₆ is phenyl optionally substituted with any combination of one to three halogen atoms, one to three C₁-C₈ alkyl groups, one to three C₁-C₆alkoxy groups, one C₁-C₄alkoxy-C₁-C₄alkyl group, one C₃-C₇cycloalkyl group, one or two C₁-C₆haloalkyl groups, one C₁-C₄alkylthio group, one cyano group, one nitro group, one C(O)R₇ group, one CO₂R₈, group, one or two X₄R₉ groups, one CH=CHR₁₀ group, one CH₂CH(R₁₁)R₁₂ group or one N(R₁₃) SO₂R₄ group;

R₇, R₁₃, R₁₄, R₁₆, R₁₇, R₁₈, R₂₂, R₂₃, R₂₆, R₂₇, R₂₉, and R₃₀ are each independently hydrogen, C₁-C₆alkyl, C₁-C₆haloalkyl, C₃-C₇cycloalkyl, C₃-C₆alkenyl, C₃-C₆alkynyl, benzyl or phenyl;

R₈, R₂₁, R₂₅ and R₂₈ are each independently hydrogen, C₁-C₁₀alkyl, C₁-C₈haloalkyl, C₃-C₇cycloalkyl, C₃-C₇halocycloalkyl, C₃-C₁₀alkenyl, C₃-C₈haloalkenyl, C₅-C₈cycloalkenyl, C₅-C₈halocycloalkenyl, C₃-C₈alkynyl, C₃-C₈haloalkynyl, benzyl, phenyl, furfuryl, pyridyl, thienyl, oximino, or an alkali metal, alkaline earth metal, manganese, copper, zinc, cobalt, silver, nickel, ammonium or organic ammonium cation;

R₉ is hydrogen, Si(R₁₅)₃, C(O)NR₁₆R₁₇, C(O)R₁₈, SO₂R₁₉, or C₁-C₁₀alkyl, C₃-C₁₀alkenyl, C₅-C₈cycloalkenyl, C₃-C₈acycloalkyl, C₃-C₁₀alkynyl, phenyl or benzyl, each of which may optionally be substituted with one to three halogen atoms, one hydroxyl, one benzyloxy, one C₁-C₆ alkoxy group, one phenoxy group, one OC(O)R₂₀ group, one CO2R21 group, one C(O)R₂₂ group, one C(O)R₂₃R₂₄ group or one cyano group;

R₁₀ and R₁₂ are each independently hydrogen, C₁-C₆alkyl, CO₂R₂₅, C(O)R₂₆ or C(O)NR₂₇R₂₈;

R₁₁ is hydrogen, halogen, C₁-C₆alkyl, C₁-C₆haloalkyl, C₃-C₇cycloalkyl, C₃-C₆alkenyl, C₃-C₆alkynyl, benzyl, phenyl or cyano;

R₁₉ is C₁-C₆alkyl, C₁-C₆haloalkyl, C₃-C₇cycloalkyl, C₃-C₆alkenyl, C₃-C₆alkynyl, benzyl, phenyl or cyano;

R₂₄ is hydrogen, C₁-C₆alkyl, C₁-C₆alkoxy, C₁-C₆haloalkyl, C₃-C₇cycloalkyl, C₃-C₆alkenyl, C₃-C₆alkynyl, SO₂R₂₉, C(O)R₃₀, benzyl or phenyl;

R₁₅ and R20 are each independently C₁-C₆alkyl, C₃-C₆alkenyl, C₃-C₆alkynyl, benzyl or phenyl; and

X₄ is O, S or NH.

This invention also relates to compositions containing those compounds and methods for using those compounds and compositions. Advantageously, it has been found that the compounds of the present invention, and compositions containing them, are useful for the control of undesirable plant species. The compounds of the present invention are especially useful for the control of undesirable plant species in the presence of crop plants.

Finally this invention provides intermediates useful in the preparation of the compound of formula I.

### DETAILED DESCRIPTION OF THE INVENTION

Surprisingly, it has been discovered that the formula I compounds of this invention are more effective and/or more selective herbicidal agents than the corresponding compounds exemplified in U.S. Pat. No. 5,523,277 wherein Q is Q₆. In addition, we have found that the formula I compounds of the present invention are particularly useful for the selective control of undesirable plant species in the presence of crops such as corn, wheat, rice and soybeans.

The herbicidal compounds of the present invention are represented by structural formula I.

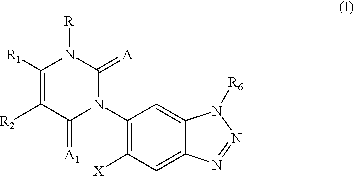

wherein R, R₁, R₂, R₆, A, A₁ and X are as defined above.

Preferred compounds of the invention are those compounds of formula I wherein

R is C₁-C₃alkyl or amino;

R₁ is C₁-C₃haloalkyl;

R₂ is hydrogen;

X is halogen;

A and A₁ are O;

R₆ is phenyl optionally substituted with any combination of one to three halogen atoms, one to three C₁-C₈alkyl groups, one to three C₁-C₆alkoxy groups, one C₁-C₄alkoxy-Cₗ-C₄alkyl group, one C₃-C₇cycloalkyl group, one or two C₁-C₆haloalkyl groups, one C₁-C₄alkylthio group, one cyano group, one nitro group, one C(O)R₇ group, one CO₂R₈ group, one or two X₄R₉groups, one CH=CHR₁₀ group, one CH₂CH(R₁₁)R₁₂ group or one N(R₁₃) SO₂R₁₄ group;

R₇, R₁₃, R₁₄, R₁₆, R₁₇, R₁₈, R₂₂, R₂₃, R₂₆, R₂₇, R₂₉ and R₃₀ are each independently hydrogen, C₁-C₆alkyl, C₁-C₆haloalkyl, C₃-C₇cycloalkyl, C₃-C₆alkenyl, C₃-C₆alkynyl, benzyl or phenyl;

R₈, R₂₁, R₂₅ and R₂₈ are each independently hydrogen, C₁-C₁₀alkyl, C₁-C₈haloalkyl, C₃-C₇cycloalkyl, C₃-C₇halocycloalkyl, C₃-C₁₀alkenyl, C₃-C₈haloalkenyl, C₅-C₈cycloalkenyl, C₅-C₈halocycloalkenyl, C₃-C₈alkynyl, C₃-C₈haloalkynyl, benzyl, phenyl, furfuryl, pyridyl, thienyl, oximino, or an alkali metal, alkaline earth metal, manganese, copper, zinc, cobalt, silver, nickel, ammonium or organic ammonium cation;

R₉ is hydrogen, Si(R₁₅)₃, C(O)NR ₁₆R₁₇, C(O)R₁₈, SO₂R₁₉, or C₁-C₁₀alkyl, C₃-C₁₀alkenyl, C₅-C₈cycloalkenyl, C₃-C₈cycloalkyl, C₃-C₁₀alkynyl, phenyl or benzyl, each of which may optionally be substituted with one to three halogen atoms, one hydroxyl, one benzyloxy, one C₁-C₆ alkoxy group, one phenoxy group, one OC(O)R₂₀ group, one CO2R21 group, one C(O)R₂₂ group, one C(O)R₂₃R₂₄ group or one cyano group;

R₁₀ and R₁₂ are each independently hydrogen, C₁-C₆alkyl, CO₂R₂₅, C(O)R₂₆ or C(O)NR₂₇R₂₈;

R₁₁ is hydrogen, halogen, C₁-C₆alkyl, C₁-C₆haloalkyl, C₃-C₇cycloalkyl, C₃-C₆alkenyl, C₃-C₆alkynyl, benzyl, phenyl or cyano;

R₁₉ is C₁-C₆alkyl, C₁-C₆haloalkyl, C₃-C₇cycloalkyl, C₃-C₆alkenyl, C₃-C₆alkynyl, benzyl, phenyl or cyano;

R₂₄ is hydrogen, C₁-C₆alkyl, C₁-C₆alkoxy, C₁-C₆haloalkyl, C₃-C₇cycloalkyl, C₃-C₆alkenyl, C₃-C₆alkynyl, SO₂R₂₉, C(O)R₃₀, benzyl or phenyl;

R₁₅ and R₂₀ are each independently C₁-C₆alkyl, C₃-C₆alkenyl, C₃-C₆alkynyl, benzyl or phenyl; and

X₄ is O.

More preferred are those compounds of formula I wherein

R is methyl or amino;

R₁ is trifluoromethyl;

R₂ is hydrogen;

X is fluorine;

R₆ is phenyl optionally substituted with any combination of one to three halogen atoms, one to three Cₗ-C₄alkyl groups, one to three C₁-C₄alkoxy groups, one methoxymethyl group, one C₃-C₇cycloalkyl group, or one or two X₄R₉ groups;

R₉ is C₁-C₃alkyl, C₃-C₆alkenyl, C₃-C₈cycloalkyl, C₃-C₆alkynyl, each of which may optionally be substituted with one halogen atom, one C₁-C₃ alkoxy group, one OC(O)R₂₀ group, one CO₂R₂₁ group, one C(O)R₂₂ group or one C(O)NR₂₃R₂₄ group;

R₂₀ is C₁-C₄alkyl, C₃-C₆alkenyl, C₃- C₆alkynyl, benzyl or phenyl;

R₂₁ is hydrogen, C₁-C₄alkyl, C₃-C₇cycloalkyl, C₃-C₆alkenyl, C₃-C₆alkynyl, benzyl, phenyl, furfuryl, pyridyl, thienyl, oximino, or an alkali metal, alkaline earth metal, manganese, copper, zinc, cobalt, silver, nickel, ammonium or organic ammonium cation;

R₂₂ R₂₃ and R₂₄ are each independently hydrogen, C₁-C₄alkyl, C₁-C₄haloalkyl, C₃-C₇cycloalkyl, C₃-C₆alkenyl, C₃-C₆alkynyl, benzyl or phenyl; and

X₄ is O.

Most preferred are compounds selected from the group consisting of

3-[1-(4-ethylphenyl)-5-fluoro-1H-1,2,3-benzotriazol-6-yl ]-1-methyl-6-(trifluoromethyl)-2,4(1H,3H)-pyrimidinedione;

3-[5-fluoro-1-(2-methoxy-5-methylphenyl)-1H-1,2,3-benzotriazol-6-yl]-1-methyl-6-(trifluoromethyl)-2,4(1H,3H)-pyrimidinedione;

methyl {2-[5-fluoro-6-(3-methyl-2,6-dioxo-4-(trifluoromethyl)-3,6-dihydro-1(2H)-pyrimidinyl)-1H-1,2,3-benzotriazol-1-yl]-4-methylphenoxy}acetate; and

3-[1-(3-ethylphenyl)-5-fluoro-1H-1,2,3-benzotriazol-6-yl]-1- methyl-6-(trifluoromethyl)-2,4(1H,3H)-pyrimidinedione.

The term halogen as used in the specification and claims designates chlorine, fluorine, bromine or iodine. The terms haloalkyl and haloalkenyl designate an alkyl group, CₙH₂ₙ₊₁, or an alkenyl group, CₙH₂ₙ, which contain from one halogen atom to 2n+1, or 2n, halogen atoms, respectively. Similarly, the term haloalkoxy designates a OCₙH₂ₙ₊₁ group which contains from one to 2n+1 halogen atoms. In all cases, the halogen atoms may be the same or different.

The Formula I compounds of the present invention are effective herbicidal agents useful for the control of a wide variety of undesirable plant species and for controlling weeds native to both dry land and wet land areas. The inventive compounds are effective in controlling the above-said plants when applied to the foliage thereof or to the soil or water containing seeds or other propagating organs thereof such as stolons, tubers or rhizomes, at rates of from 0.01 kg/ha to 1 kg/ha.

Advantageously, it has been found that the compounds of the invention are selective in the presence of soybeans or cereal crops such as corn, wheat and rice when applied preemergence or postemergence.

Beneficially, the formula I compounds may be used for the selective control of undesirable plant species in transplanted rice culture by applying a herbicidally effective amount of a formula I compound to the soil or water containing the seeds or other propagating organs of said undesirable plant species after the rice has been transplanted.

While the compounds of this invention are effective for controlling undesirable plant species when employed alone, they may also be used in combination with or in conjunction with one or more biological chemicals, including herbicides.

The compounds of this invention may be applied to the foliage of undesirable plant species or to the soil or water containing seeds or other propagating organs thereof in the form of a solid or liquid herbicidal composition, comprising a herbicidally effective amount of the desired compound dispersed or dissolved in an agronomically acceptable, inert solid or liquid carrier. The compositions may be applied as preemergence or postemergence treatments.

The formula I compounds of the present invention may be formulated as emulsifiable concentrates, wettable powders, granular formulations, suspension concentrates, flowable concentrates and the like.

Formula I compounds wherein A and A₁, are O may be prepared, as shown in Flow Diagram I, by reacting an aminobenzotriazole of formula II with a 2-dialkylamino-6H-1,3-oxazin-6-one of formula III in the presence of an organic acid such as acetic acid to form an intermediate compound of formula IV and reacting the formula IV compound with an electrophile of formula V in the presence of a base such as potassium carbonate.

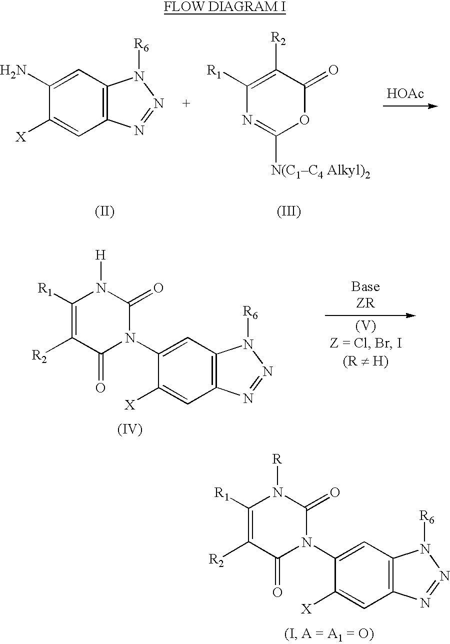

Alternatively, intermediate compounds of formula IV may be prepared by reacting an aminobenzotriazole of formula II with phosgene or an equivalent thereof to form an intermediate isocyanate of formula VI, followed by reaction of said isocyanate VI with an amino-crotonate of formula VII in the presence of a base, as depicted in Flow Diagram II.

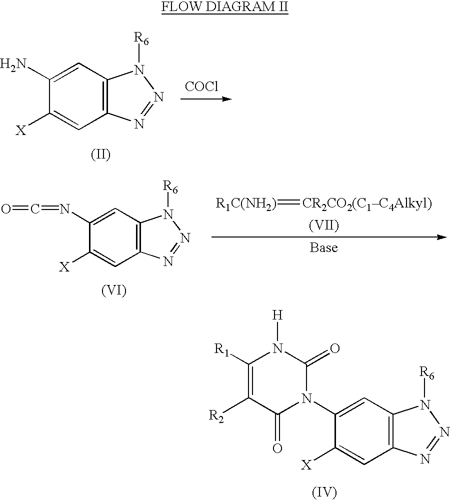

Aminobenzotriazoles of formula II may be prepared as described in U.S. Pat. No. 5,523,277.

Formula I compounds wherein A is O and A₁ is S may be prepared, as shown in Flow Diagram III, by reacting a formula I compound wherein A and A₁ are O with Lawesson's Reagent (2,4-bis(4-methoxyphenyl)-1,3-dithia-2,4-diphosphetane-2,4-disulfide) in the presence of a base such as sodium hydrogen carbonate.

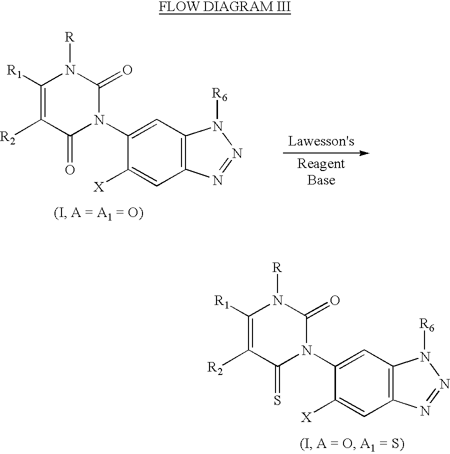

Formula I compounds wherein A and A₁ are S may be prepared, as shown in Flow Diagram IV, by reacting a formula I compound wherein A is O and A₁ is S with Lawesson's Reagent in the presence of a base.

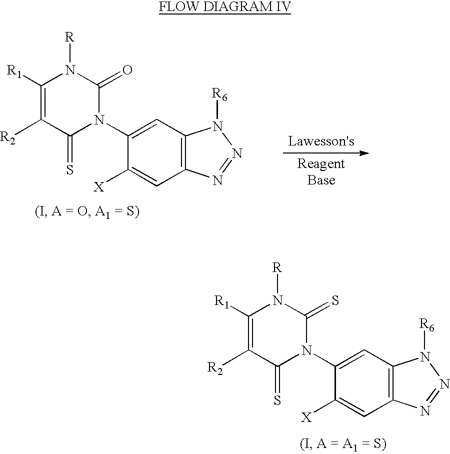

This invention also provides intermediate compounds of formula VIII

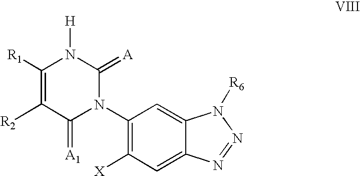

wherein R₁, R₂, R₆, A, A₁ and X are as described hereinabove for formula I.

Preferred compounds of formula VIII are those wherein

R₁ is C₁-C₃haloalkyl;

R₂ is hydrogen;

X is halogen;

A and A₁ are O;

R₆ . is phenyl optionally substituted with any combination of one to three halogen atoms, one to three C₁-C₈alkyl groups, one to three C₁-C₆alkoxy groups, one C₁-C₄alkoxy-C₁-C₄alkyl group, one C₃-C₇cycloalkyl group, one or two C₁-C₆haloalkyl groups, one C₁-C₄alkylthio group, one cyano group, one nitro group, one C(O)R₇ group, one CO₂R₈ group, one or two X₄R₉ groups, one CH=CHR₁₀ group, one CH₂CH(R₁₁)R₁₂ group or one N(R₁₃) SO₂R₁₄ group;

R₇, R₁₃, R₁₄, R₁₆, R₁₇, R18 R₂₂ R₂₃ , R₂₆, R₂₇, R₂₉ and R₃₀ are each independently hydrogen, C₁-C₆alkyl, C₁-C₆haloalkyl, C₃ -C₇cycloalkyl, C₃-C₆alkenyl, C₃-C₆alkynyl, benzyl or phenyl;

R₈, R₂₁, R₂₅ and R₂₈ are each independently hydrogen, C₁-C₁₀alkyl, C₁-C₈haloalkyl, C₃-C₇cycloalkyl, C₃-C₇halocycloalkyl, C₃-C₁₀alkenyl, C₃-C₈haloalkenyl, C₅-C₈cycloalkenyl, C₅-C₈halocycloalkenyl, C₃-C₈alkynyl, C₃-C₈haloalkynyl, benzyl, phenyl, furfuryl, pyridyl, thienyl, oximino, or an alkali metal, alkaline earth metal, manganese, copper, zinc, cobalt, silver, nickel, ammonium or organic ammonium cation;

R₉ is hydrogen, Si(R₁₅)₃, C(O)NR₁₆R₁₇, C(O)R₁₈, SO₂R₁₉, or C₁-C₁₀alkyl, C₃-C₁₀alkenyl, C₅-C₈cycloalkenyl, C₃-C₈cycloalkyl, C₃-C₁₀alkynyl, phenyl or benzyl, each of which may optionally be substituted with one to three halogen atoms, one hydroxyl, one benzyloxy, one C₁-C₆ alkoxy group, one phenoxy group, one OC(O)R₂₀ group, one CO₂R₂₁ group, one C(O)R₂₂ group, one C(O)R₂₃R₂₄ group or one cyano group;

R₁₀ and R₁₂ are each independently hydrogen, C₁-C₆alkyl, CO₂R₂₅, C(O)R₂₆ or C(O)NR₂₇R₂₈;

R₁₁ is hydrogen, halogen, C₁-C₆alkyl, C₁-C₆haloalkyl, C₃-C₇cycloalkyl, C₃-C₆alkenyl, C₃-C₆alkynyl, benzyl, phenyl or cyano;

R₁₉ is C₁-C₆alkyl, C₁-C₆haloalkyl, C₃-C₇cycloalkyl, C₃-C₆alkenyl, C₃-C₆alkynyl, benzyl, phenyl or cyano;

R₂₄ is hydrogen, C₁-C₆alkyl, C₁-C₆alkoxy, C₁-C₆haloalkyl, C₃-C₇cycloalkyl, C₃-C₆alkenyl, C₃-C₆alkynyl, SO₂R₂₉, C(O)R₃₀, benzyl or phenyl;

R₁₅ and R₂₀ are each independently C₁-C₆alkyl, C₃-C₆alkenyl, C₃-C₆alkynyl, benzyl or phenyl; and

X₄ is O.

More preferred compounds of formula VIII are those wherein

R₁ is trifluoromethyl;

R₂ is hydrogen;

X is fluorine;

Y is R₆;

R₆ is phenyl optionally substituted with any combination of one to three halogen atoms, one to three C₁-C₄alkyl groups, one to three C₁-C₄alkoxy groups, one methoxymethyl group, one C₃-C₇cycloalkyl group, or one or two X₄R₉ groups;

R₉ C₁-C₃alkyl, C₃-C₆alkenyl, C₃-C₈cycloalkyl, C₃-C₆alkynyl, each of which may optionally be substituted with one halogen atom, one C₁-C₃ alkoxy group, one OC(O)R₂₀ group, one CO₂R₂₁ group, one C(O)R₂₂ group or one C(O)NR₂₃R₂₄ group;

R₂₀ is C₁-C₄alkyl, C₃-C₆alkenyl, C₃-C₆alkynyl, benzyl or phenyl;

R₂₁ hydrogen, C₁-C₄alkyl, C₃-C₇cycloalkyl, C₃-C₆alkenyl, C₃-C₆alkynyl, benzyl, phenyl, furfuryl, pyridyl, thienyl, oximino, or an alkali metal, alkaline earth metal, manganese, copper, zinc, cobalt, silver, nickel, ammonium or organic ammonium cation;

R₂₂ R₂₃ and R₂₄ are each independently hydrogen, C₁-C₄alkyl, C₁-C₄haloalkyl, C₃-C₇cycloalkyl, C₃-C₆alkenyl, C₃-C₆alkynyl, benzyl or phenyl; and

X₄ is O.

Most preferred compounds of formula VIII are those selected from the group consisting of

3-[1-(4-ethylphenyl)-5-fluoro-1H-1,2,3-benzotriazol-6-yl]-6-(trifluoromethyl)-2,4(1H,3H)-pyrimidinedione;

3-[5-fluoro-1-(2-methoxy-5-methylphenyl)-1H-1,2,3-benzotriazol-6-yl]-6-(trifluoromethyl)-2,4(1H,3H)-pyrimidinedione; and

3-[1-(3-ethylphenyl)-5-fluoro-1H-1,2,3-benzotriazol-6-yl]-6-(trifluoromethyl)-2,4(1H,3H)-pyrimidinedione.

In order to facilitate a further understanding of the invention, the following examples are presented to illustrate more specific details thereof. The invention is not to be limited thereby except as defined in the claims. The terms ¹H NMR ¹³C NMR, and ¹⁹F NMR designate proton, carbon and fluorine nuclear magnetic spectroscopy respectively, and the terms IR and MS designate infrared spectrometry and mass spectroscopy, respectively.

### EXAMPLE 1

### Preparation of 3-[5-Flouro-1-(2-Methoxyphenyl)-1H-1,2,3- Benzotriazol-6-yl]-6-(Trufluoromethyl)-2,4(1H, 3H)-Pyrimidinedione

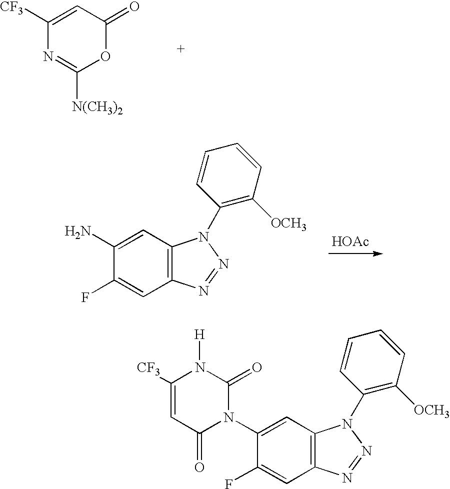

To a solution of 5-fluoro-1-(2-methoxyphenyl)-1H-1,2,3-benzotriazol-6-amine (1.0 g, 0.0039 mol) in acetic acid (25 mL) is added 2-(dimethylamino)-4-(trifluoromethyl)-6H-1,3-oxazine-6-one (1.1 g, 0.005 mol), and the resulting solution refluxed for ca. 1 hour. The reaction mixture is poured into water, and the resulting solid filtered and dried to afford the title compound as a light tan solid (1.4 g, 87%, mp 207-211° C.) which is characterized by IR and NMR spectral analyses.

### EXAMPLES 2-15

### Preparation of 6-(Trifluoromethyl)-2,4(1H,3H)-Pyrimidinediones

Using essentially the same procedure as described in Example 1 and the appropriate amine, the following compounds are obtained.

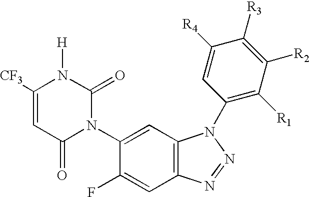

| Ex. No. | R₁ | R₂ | R₃ | R₄ | mp (° C.) |
|---|---|---|---|---|---|
| 2 | OCH₃ | H | H | CH₃ | 145-150 |
| 3 | H | H | CH₂CH₃ | H | 160-164 |
| 4 | H | H | CH₃ | H | 228-232 |
| 5 | CH₃ | H | H | H | 145-150 |
| 6 | H | CH₃ | CH₃ | H | 222-227 |
| 7 | CH₃ | H | H | OCH₃ | 148-151 |
| 8 | CH₃ | H | OCH₃ | H | 104-106 |
| 9 | CH₂CH₃ | H | H | H | 168-175 |
| 10 | CH₂OCH₃ | H | H | H | 91-93 |
| 11 | H | CH₂OCH₃ | H | H | 98-102 |
| 12 | H | H | OCH₃ | H | 7200 |
| 13 | H | CH₂CH₃ | H | H | 160 |
| 14 | OCH₃ | H | H | Cl | 123 |
| 15 | H | H | OPh | H | 95-105 |

### EXAMPLE 16

### Preparation of 3-[5-Fluoro-1-(2-Methoxyphenyl)-1H-1,2,3-Benzotriazol-6-yl]-1-Methyl-6-(Trifluoromethyl)-2,4(1H,3H)-Pyrmidinedione

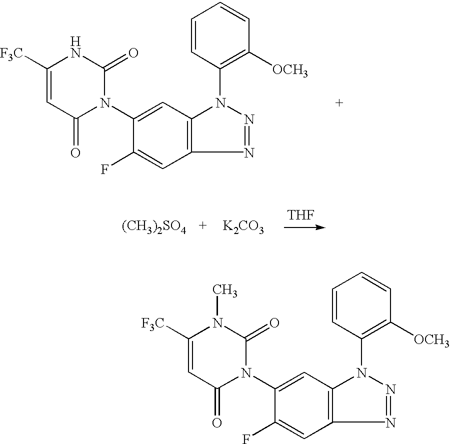

To a solution of 3-[5-fluoro-1-(2-methoxyphenyl)-1H-1, 2, 3-benzotriazol-6-yl]-6-(trifluoromethyl)-2, 4(1H, 3H)-pyrimidinedione (1.6 g, 0.0038 mol) in dry tetrahydrofuran (35 mL) is added potassium carbonate (0.07 g, 0.005 mol) followed by dimethylsulfate (0.48 g, 0.005 mol). After 90 minutes at reflux, additional dimethylsulfate (0.02 mL, 0.0002 mol) is added. The reaction mixture is then cooled, poured into water and extracted with ethyl acetate. The combined ethyl acetate extracts are dried and concentrated in vacuo, and the residue is chromatographed on silica gel eluting with hexane: ethyl acetate (75:25 to 50:50). The title compound is obtained as a white solid (1.3 g, 81%, mp 123-126° C.) which is identified by IR, NMR and mass spectral analyses.

### EXAMPLES 17-30

### Preparation of 1-Methyl-6-(Trifluoromethyl)-2,4(1H,3H)-Pyrimidinediones

Using essentially the same procedure as described in Example 16, the following compounds are obtained.

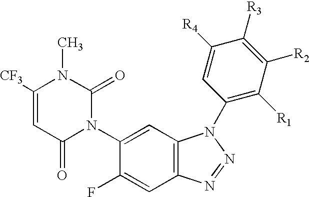

| Ex. No. | R₁ | R₂ | R₃ | R₄ | mp (° C.) |
|---|---|---|---|---|---|
| 17 | H | H | CH₂CH₃ | H | 189-192 |
| 18 | OCH₃ | H | H | CH₃ | 175-181 |
| 19 | H | H | CH₃ | H | 167-170 |
| 20 | CH₃ | H | H | H | 66-68 |
| 21 | H | H | CH (CH₃) ₂ | H | 163-166 |
| 22 | CH₃ | H | H | OCH₃ | 64-67 |
| 23 | H | CH₃ | CH₃ | H | 77-87 |
| 24 | CH₃ | H | OCH₃ | H | 75-77 |
| 25 | CH₂CH₃ | H | H | H | 77-87 |
| 26 | CH₂OCH₃ | H | H | H | 68-70 |
| 27 | H | H | OCH₃ | H | 216-220 |
| 28 | H | CH₂OCH₃ | CH₂CH₃ | H | 62-66 |
| 29 | H | CH₂CH₃ | H | H | 52-65 |
| 30 | OCH₃ | H | H | Cl | 96-97 |

### EXAMPLE 31

### Preparation of 3-[5-Fluoro-1-(2-Hydroxy-5-Methylphenyl)-1H-1,2,3-Benzotriazol-6-yl]-1-Methyl-6-(Trifluoromethyl)-2,4(1H,3H)-(Pyrimidinedione)

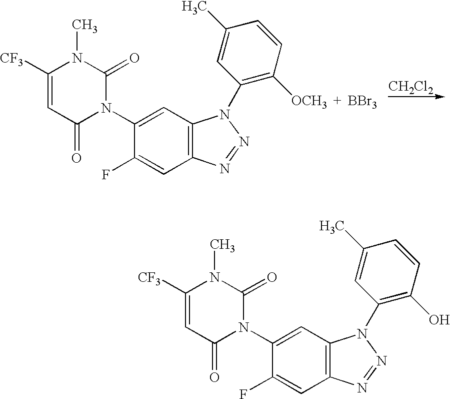

To a solution of 3-[5-fluoro-1-(2-methoxy-5-methylphenyl)-1H-1,2,3-benzotriazol-6-yl]-1-methyl-6-(trifluoromethyl)-2,4(1H,3H)-pyrimidinedione (1.0 g, 0.0022 mol) in dichloromethane at 10° C. is added a solution of boron tribromide (1 M, 13 mL, 0.013 mol) in dichloromethane, and the resulting solution refluxed overnight. An additional quantity of boron tribromide (5 mL, 0.005 mol) is then added and refluxing continued for 8 hours. The reaction mixture is cooled to room temperature, stirred for several hours, and then poured into water and neutralized with saturated sodium bicarbonate. The organic layer is separated, dried over anhydrous magnesium sulfate, and concentrated in vacuo to a dark solid (0.75 g). The residue is purified by flash chromatography on silica gel eluting with hexane: ethyl acetate (50:50) to afford a solid which, after titration with etherhexane, gives the title compound as an off-white solid (0.44 g, 46%, mp 248-249° C.), which is identified by NMR and mass spectral analyses.

### EXAMPLES 32-33

### Preparation of 3-[5-Fluoro-1-(2-1-(Hydroxyphenyl)-1H-1,2,3-Benzotriazol-6-yl]-1-Methyl-6-(Trifluoromethyl)-2,4(1H,3H)-Pyrimidinediones

In essentially the same manner as described in example 32, using the appropriate substrate, the following compounds are prepared.

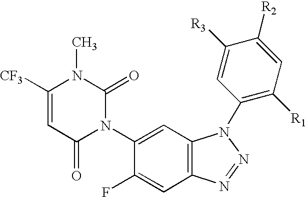

| Ex. No. | R₁ | R₂ | R₃ | mp (° C.) |
|---|---|---|---|---|
| 32 | CH₃ | H | OH | 108-110 |
| 33 | CH₃ | OH | H | >250 |

### EXAMPLE 34

### Preparation of Methyl {2-[5-Fluoro-6-(3-Methyl-2,6-Dioxo-4-(Trifluoromethyl) -3,6-Dihydro-1(2H)-Pyrimidinyl)-1H-1,2,3H)-Benzotriazol-1-yl]-4-Methylphenoxy}Acetate

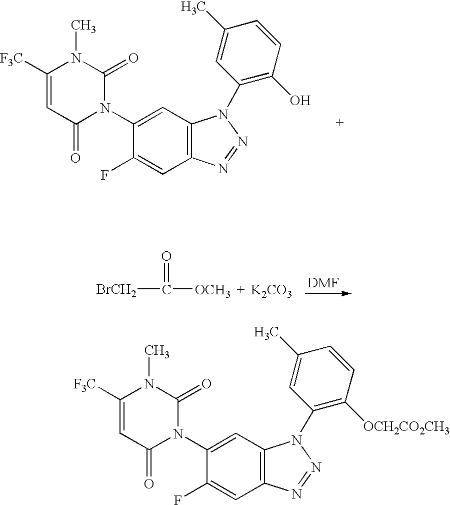

3-[5-fluoro-1-(2-hydroxy-5-methylphenyl)-1H-1,2,3-benzotriazol-6-yl]-1-methyl-6- (trifluoromethyl) -2,4 (1H,3H)-pyrimidinedione (0.5 g, 0.0011 mol) is added to dry dimethylformamide (5 mL) followed by methyl bromoacetate (0.18 g. 0.0012 mol) and potassium carbonate (0.18 g, 0.0013 mol) . After stirring overnight at room temperature, the reaction mixture is poured into water to give, after drying, the title compound as a white solid (0.4 g, 71%, mp 95-98° C.) which is identified by IR, NMR and mass spectral analyses.

### EXAMPLES 35-45

### Preparation of O-Alkylated 3-[1-(Hydroxyphenyl)-1H-1,2,3-Benzotriazol-6-yl]-1-Methyl-6-(Trifluoromethyl)2,4(1H, 3H)-Pyrimidinediones

Using essentially the same procedure as described in Example 34, on the appropriate phenols, the following compounds are obtained.

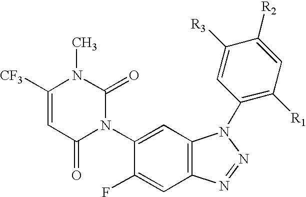

| Ex. No. | R₁ | R₂ | R₃ | mp (° C.) |
|---|---|---|---|---|
| 35 | OCH₂CO₂CH₃ | H | H | 159-164 |
| 36 | CH₃ | H | OCH₂CO₂CH₂C≡CH | 185-187 |
| 37 | CH₃ | H | OCH₂CO₂H | 84 |
| 38 | CH₃ | H | OCH₂CO₂CH₃ | 72-74 |
| 39 | CH₃ | OCH₂CO₂CH₃ | H | 77-80 |
| 40 | OCH₂CO₂CH₂C≡CH | H | CH₃ | 79-84 |
| 41 | OCH(CH₃)CO₂CH₃ | H | CH₃ | 100-105 |
| 42 | OCH₂CH═CH₂ | H | CH₃ | 133-136 |
| 43 | CH₃ | OCH₂CH═CH₂ | H | 72 |
| 44 | H | OCH₂CO₂H | H | >220 |
| 45 | H | OCH₂CO₂CH₃ | H | 79-85 |

### EXAMPLE 46

### Postemergence Herbicidal Evaluation of Test Compounds

In this evaluation, seedling plants are grown in jiffy flats for about two weeks. The test compounds are dispersed in 80/20 acetone/water mixtures containing 1.0% SUN-IT® II, a methylated seed oil, in sufficient quantities to provide the equivalent of about 0.006 kg to 0.400 kg per hectare of active compound when applied to the plants through a spray nozzle operating at 40 psi for a predetermined time. After spraying, the plants are placed on greenhouse benches and are cared for in accordance with conventional greenhouse procedures. Approximately two - three weeks after treatment, the seedling plants are examined and rated according to the rating system set forth below. Data obtained are reported in Table I below. Where more than one test is involved for a given compound, the data are averaged.

HERBICIDE RATING SCALE

| | % Control |
|---|---|
| Rating | Compared to Check |
| 9 | 100 |
| 8 | 91-99 |
| 7 | 80-90 |
| 6 | 65-79 |
| 5 | 45-64 |
| 4 | 30-44 |
| 3 | 16-29 |
| 2 | 6-15 |
| 1 | 1-5 |
| 0 | 0 |

PLANT SPECIES EMPLOYED IN HERBICIDAL EVALUATIONS

| Header | | |
|---|---|---|
| Abbr. | Common Name | Scientific Name |
| ABUTH | Velvetleaf | Abutilon theophrasti, |
| | | Medic. |
| AMBEL | Ragweed, Common | Ambrosia artemisifolia, |
| | | L. |
| CASOB | Sicklepod | Cassia obtusifolia, L. |
| CHEAL | Lambsquarters, Common | Chenopodium album, L. |
| ECHOG | Barnyardgrass | Echinochloa crus-galli, |
| | | (L.) Beau |
| GALAP | Galium | Galium aparine |
| IPOHE | Morningglory, Ivyleaf | Ipomoea hederacea, (L.) |
| | | Jacq. |
| SETVI | Foxtail, Green | Setaria viridis, |
| | | (L.) Beau |
| GLXMAW | Soybean, Williams | Glycine max, (L.) Merr. |
| | | cv Williams |
| TRZAW | Wheat, Winter, | Triticum aestivum, L. |
| | Unspecified | (Winter) |
| TRZAWR | Wheat, Winter, | Triticum aestivum, |
| | cv Riband | cv Riband |
| ORYSAT | Rice, Tebonnet | Oryza sativa, (L.) |
| ZEAMX | Corn, Field | Zea mays, L. (Sammel- |
| | | Bezeichnung) |

TABLE I
POSTEMERGENCE HERBICIDAL EVALUATION

| Ex | Rate | | | | | | | | | | | | |
|---|---|---|---|---|---|---|---|---|---|---|---|---|---|
| No. | (kg/ha) | ABUTH | AMBEL | CASOB | CHEAL | GALAP | IPOHE | ECHCG | SETVI | GLXMAW | ORYSAT | TRZAWR | ZEAMX |
| 16 | 0.062 | 9.0 | 9.0 | 9.0 | 8.0 | 4.0 | 9.0 | 5.0 | 9.0 | 7.5 | 4.0 | 5.5 | 6.0 |
| | 0.032 | 9.0 | 9.0 | 6.0 | 6.0 | 3.0 | 9.0 | 3.0 | 7.0 | 6.0 | 3.0 | 3.5 | 6.0 |
| | 0.016 | 9.0 | 9.0 | 6.0 | 5.0 | 3.0 | 5.0 | 2.0 | 5.0 | 5.0 | 3.0 | 3.0 | 6.0 |
| | 0.008 | 9.0 | 2.0 | 6.0 | 5.0 | 0.0 | 5.0 | 2.0 | 2.0 | 5.0 | 2.5 | 2.5 | 5.5 |
| 17 | 0.062 | 9.0 | 9.0 | 6.0 | 9.0 | 7.0 | 9.0 | 5.0 | 8.0 | 7.0 | 4.0 | 5.0 | 5.5 |
| | 0.032 | 9.0 | 8.0 | 5.0 | 7.0 | 4.0 | 9.0 | 1.0 | 3.0 | 5.5 | 3.5 | 4.0 | 5.5 |
| | 0.016 | 9.0 | 6.0 | 3.0 | 6.0 | 4.0 | 9.0 | 0.0 | 1.0 | 5.5 | 3.0 | 3.5 | 5.5 |
| | 0.008 | 9.0 | 5.0 | 1.0 | 9.0 | 2.0 | 9.0 | 0.0 | 1.0 | 5.5 | 3.0 | 3.5 | 5.0 |
| 18 | 0.062 | 9.0 | 9.0 | 7.0 | 9.0 | 5.0 | 9.0 | 4.0 | 9.0 | 6.0 | 5.5 | 4.5 | 6.0 |
| | 0.032 | 9.0 | 6.0 | 6.0 | 9.0 | 5.0 | 9.0 | 2.0 | 9.0 | 6.5 | 5.5 | 5.5 | 6.0 |
| | 0.016 | 9.0 | 6.0 | 7.0 | 7.0 | 3.0 | 9.0 | 2.0 | 5.0 | 4.5 | 4.0 | 4.5 | 5.5 |
| | 0.008 | 9.0 | 1.0 | 0.0 | 1.0 | 1.0 | 9.0 | 0.0 | 0.0 | 4.5 | 3.5 | 3.5 | 4.5 |
| 19 | 0.062 | 9.0 | 5.0 | 2.0 | 5.0 | 4.0 | 9.0 | 0.0 | 3.0 | 7.5 | 4.0 | 4.5 | 5.5 |
| | 0.032 | 9.0 | 4.0 | 2.0 | 3.0 | 4.0 | 9.0 | 0.0 | 0.0 | 6.5 | 3.0 | 4.0 | 5.0 |
| | 0.016 | 9.0 | 1.0 | 0.0 | 2.0 | 3.0 | 4.0 | 0.0 | 0.0 | 5.5 | 2.5 | 3.5 | 4.5 |
| | 0.008 | 9.0 | 0.0 | 0.0 | 1.0 | 0.0 | 4.0 | 0.0 | 0.0 | 4.5 | 2.0 | 2.5 | 4.5 |
| 20 | 0.062 | 9.0 | 3.0 | 1.0 | 5.0 | 9.0 | 9.0 | 0.0 | 0.0 | 5.5 | 1.0 | 2.5 | 5.0 |
| | 0.032 | 9.0 | 1.0 | 0.0 | 3.0 | 2.0 | 4.0 | 0.0 | 0.0 | 5.5 | 0.5 | 1.5 | 3.5 |
| | 0.016 | 9.0 | 1.0 | 0.0 | 0.0 | 0.0 | 4.0 | 0.0 | 0.0 | 5.5 | 0.5 | 1.5 | 3.5 |
| | 0.008 | 9.0 | 0.0 | 0.0 | 0.0 | 0.0 | 1.0 | 0.0 | 0.0 | 3.0 | 0.0 | 1.5 | 3.0 |
| 21 | 0.062 | 9.0 | 9.0 | 2.0 | 9.0 | 9.0 | 9.0 | 0.0 | 5.0 | 5.5 | 3.5 | 4.0 | 5.0 |
| | 0.032 | 9.0 | 9.0 | 1.0 | 9.0 | 4.0 | 6.0 | 0.0 | 5.0 | 5.5 | 3.5 | 4.0 | 4.5 |
| | 0.016 | 9.0 | 9.0 | 0.0 | 4.0 | 4.0 | 9.0 | 0.0 | 0.0 | 5.0 | 3.5 | 4.0 | 4.5 |
| | 0.008 | 9.0 | 2.0 | 0.0 | 8.0 | 2.0 | 9.0 | 0.0 | 0.0 | 4.5 | 3.0 | 3.5 | 4.5 |
| 22 | 0.062 | 9.0 | 3.0 | 0.0 | 4.0 | 5.0 | 2.0 | 0.0 | 0.0 | 5.0 | 3.0 | 4.5 | 4.5 |
| | 0.032 | 9.0 | 2.0 | 0.0 | 3.0 | 3.0 | 9.0 | 0.0 | 0.0 | 4.0 | 3.0 | 4.0 | 4.0 |
| | 0.016 | 9.0 | 0.0 | 0.0 | 9.0 | 0.0 | 2.0 | 0.0 | 0.0 | 4.5 | 2.5 | 3.5 | 4.5 |
| | 0.008 | 9.0 | 0.0 | 0.0 | 9.0 | 0.0 | 2.0 | 0.0 | 0.0 | 4.0 | 2.0 | 3.0 | 4.5 |
| 23 | 0.062 | 9.0 | 9.0 | 3.0 | 9.0 | 2.0 | 9.0 | 0.0 | 9.0 | 4.5 | 4.0 | 6.5 | 8.5 |
| | 0.032 | 9.0 | 4.0 | 0.0 | 4.0 | 2.0 | 9.0 | 0.0 | 9.0 | 4.5 | 3.5 | 5.5 | 8.0 |
| | 0.016 | 9.0 | 0.0 | 0.0 | | 2.0 | 9.0 | 0.0 | 9.0 | 4.5 | 3.0 | 4.5 | 7.0 |
| | 0.008 | 9.0 | 0.0 | 0.0 | 3.0 | 0.0 | 2.0 | 0.0 | 7.0 | 3.5 | 2.5 | 4.0 | 5.5 |
| 24 | 0.062 | 9.0 | 2.0 | 0.0 | 4.0 | 3.0 | 4.0 | 0.0 | 0.0 | 4.0 | 2.5 | 3.5 | 4.5 |
| | 0.032 | 9.0 | 0.0 | 0.0 | 3.0 | 2.0 | 4.0 | 0.0 | 0.0 | 4.0 | 2.0 | 2.5 | 3.5 |
| | 0.016 | 9.0 | 0.0 | 0.0 | 3.0 | 1.0 | 3.0 | 0.0 | 0.0 | 3.5 | 2.0 | 2.5 | 3.5 |
| | 0.008 | 3.0 | 0.0 | 0.0 | 3.0 | 0.0 | 0.0 | 0.0 | 0.0 | 3.0 | 2.0 | 2.5 | 3.5 |
| 25 | 0.062 | 9.0 | 2.0 | 1.0 | 9.0 | 4.0 | 9.0 | 0.0 | 7.0 | 6.5 | 2.0 | 3.0 | 5.0 |
| | 0.032 | 9.0 | 2.0 | 0.0 | 9.0 | 4.0 | 9.0 | 0.0 | 3.0 | 5.0 | 1.5 | 3.0 | 4.0 |
| | 0.016 | 8.0 | 0.0 | 0.0 | 9.0 | 0.0 | 3.0 | 0.0 | 0.0 | 4.5 | 1.0 | 2.5 | 3.5 |
| | 0.008 | 6.0 | 0.0 | 0.0 | 3.0 | 0.0 | 2.0 | 0.0 | 0.0 | 3.0 | 0.5 | 1.5 | 3.0 |
| 26 | 0.062 | 9.0 | 9.0 | 4.0 | | 6.0 | 9.0 | 3.0 | 5.0 | 9.0 | 4.5 | 4.0 | 6.5 |
| | 0.032 | 9.0 | 7.0 | 2.0 | 9.0 | 2.0 | 9.0 | 1.0 | 2.0 | 5.5 | 3.0 | 3.0 | 4.0 |
| | 0.016 | 9.0 | 6.0 | 2.0 | 7.0 | 1.0 | 9.0 | 1.0 | 5.0 | 5.0 | 2.0 | 2.5 | 4.0 |
| | 0.008 | 9.0 | 4.0 | 1.0 | 2.0 | 1.0 | 4.0 | 1.0 | 1.0 | 4.5 | 2.0 | 2.0 | 3.5 |
| 27 | 0.032 | 9.0 | 9.0 | 6.0 | 9.0 | 9.0 | 9.0 | 2.0 | 7.0 | 6.0 | 4.5 | 4.5 | 5.5 |
| | 0.016 | 9.0 | 9.0 | 1.0 | 7.0 | 4.0 | 9.0 | 1.0 | 4.0 | 6.0 | 4.5 | 4.0 | 5.0 |
| | 0.008 | 9.0 | 5.0 | 1.0 | 4.0 | 1.0 | 9.0 | 1.0 | 7.0 | 5.0 | 2.5 | 2.0 | 4.5 |
| 28 | 0.062 | 9.0 | 9.0 | 3.0 | 9.0 | 8.0 | 9.0 | 1.0 | 9.0 | 6.5 | 5.5 | 5.5 | 7.5 |
| | 0.032 | 9.0 | 9.0 | 1.0 | 7.0 | 6.0 | 9.0 | 1.0 | 7.0 | 6.0 | 3.0 | 4.5 | 6.0 |
| | 0.016 | 9.0 | 9.0 | 0.0 | 4.0 | 4.0 | 9.0 | 0.0 | 3.0 | 5.0 | 2.0 | 4.5 | 6.0 |
| | 0.008 | 9.0 | 9.0 | 0.0 | 2.0 | 4.0 | 9.0 | 0.0 | 0.0 | 4.0 | 0.5 | 2.5 | 3.5 |
| 29 | 0.062 | 9.0 | 9.0 | 3.0 | 9.0 | 3.0 | 9.0 | 1.0 | 9.0 | 8.0 | 3.0 | 3.0 | 5.0 |
| | 0.032 | 9.0 | 6.0 | 2.0 | 9.0 | 1.0 | 9.0 | 1.0 | 9.0 | 8.0 | 2.0 | 2.5 | 5.0 |
| | 0.016 | 9.0 | 7.0 | 2.0 | 9.0 | 1.0 | 9.0 | 1.0 | 7.0 | 6.5 | 2.0 | 2.0 | 5.0 |
| | 0.008 | 9.0 | 7.0 | 0.0 | 9.0 | 0.0 | 9.0 | 1.0 | 1.0 | 5.0 | 2.0 | 2.0 | 3.5 |
| 30 | 0.062 | 9.0 | 9.0 | 8.0 | 9.0 | 4.0 | 9.0 | 6.0 | 9.0 | 8.0 | 4.5 | 4.5 | 5.0 |
| | 0.032 | 9.0 | 9.0 | 7.0 | 9.0 | 4.0 | 9.0 | 4.0 | 9.0 | 8.0 | 4.0 | 4.0 | 5.0 |
| | 0.016 | 9.0 | 9.0 | 4.0 | 9.0 | 3.0 | 9.0 | 4.0 | 6.0 | 6.5 | 3.5 | 3.5 | 4.5 |
| | 0.008 | 9.0 | 9.0 | 3.0 | 9.0 | 1.0 | 9.0 | 2.0 | 7.0 | 6.5 | 3.5 | 3.5 | 4.5 |
| 31 | 0.062 | 4.0 | 3.0 | 0.0 | 0.0 | 0.0 | 4.0 | 3.0 | 5.0 | 5.0 | 1.5 | 3.0 | 4.5 |
| | 0.032 | 2.0 | 0.0 | 0.0 | 0.0 | 0.0 | 4.0 | 0.0 | 0.0 | 4.5 | 1.5 | 3.0 | 4.5 |
| | 0.016 | 0.0 | 0.0 | 0.0 | 0.0 | 0.0 | 2.0 | 0.0 | 0.0 | 3.5 | 1.0 | 2.0 | 3.5 |
| | 0.008 | 0.0 | 0.0 | 0.0 | 0.0 | 0.0 | 1.0 | 0.0 | 0.0 | 3.5 | 1.0 | 2.0 | 3.5 |
| 32 | 0.062 | 4.0 | 0.0 | 0.0 | 0.0 | 0.0 | 0.0 | 0.0 | 0.0 | 3.0 | 2.0 | 3.0 | 2.5 |
| | 0.032 | 0.0 | 0.0 | 0.0 | 0.0 | 0.0 | 0.0 | 0.0 | 0.0 | 3.0 | 1.5 | 2.5 | 2.5 |
| | 0.016 | 0.0 | 0.0 | 0.0 | 0.0 | 0.0 | 0.0 | 0.0 | 0.0 | 2.5 | 1.5 | 2.5 | 2.5 |
| | 0.008 | 0.0 | 0.0 | 0.0 | 0.0 | 0.0 | 0.0 | 0.0 | 0.0 | 2.0 | 1.0 | 1.0 | 2.0 |
| 33 | 0.062 | 5.0 | 0.0 | 0.0 | 2.0 | 0.0 | 9.0 | 0.0 | 0.0 | 5.0 | 2.0 | 2.5 | 4.0 |
| | 0.032 | 3.0 | 0.0 | 0.0 | 0.0 | 0.0 | 3.0 | 0.0 | 0.0 | 4.0 | 0.5 | 2.0 | 3.5 |
| | 0.016 | 3.0 | 0.0 | 0.0 | 0.0 | 0.0 | 2.0 | 0.0 | 0.0 | 3.5 | 0.0 | 1.0 | 3.0 |
| | 0.008 | 0.0 | 0.0 | 0.0 | 0.0 | 0.0 | 0.0 | 0.0 | 0.0 | 1.5 | 0.0 | 0.5 | 3.0 |
| 34 | 0.062 | 9.0 | 9.0 | 5.0 | 9.0 | 4.0 | 9.0 | 9.0 | 9.0 | 7.0 | 7.5 | 8.0 | 9.0 |
| | 0.032 | 9.0 | 9.0 | 1.0 | 3.0 | 4.0 | 9.0 | 9.0 | 9.0 | 7.0 | 6.5 | 7.0 | 8.5 |
| | 0.016 | 9.0 | 3.0 | 0.0 | 3.0 | 3.0 | 9.0 | 9.0 | 9.0 | 6.5 | 4.0 | 5.0 | 7.5 |
| | 0.008 | 9.0 | 0.0 | 0.0 | 2.0 | 0.0 | 9.0 | 7.0 | 9.0 | 5.0 | 3.0 | 5.0 | 7.0 |
| 35 | 0.062 | 9.0 | 9.0 | 2.0 | 7.0 | 4.0 | 9.0 | 9.0 | 8.0 | 7.5 | 7.0 | 7.0 | 7.5 |
| | 0.032 | 9.0 | 9.0 | 2.0 | 6.0 | 3.0 | 9.0 | 7.0 | 9.0 | 6.0 | 6.0 | 5.5 | 5.5 |
| | 0.016 | 5.0 | 5.0 | 1.0 | 4.0 | 1.0 | 4.0 | 4.0 | 9.0 | 4.5 | 3.5 | 3.0 | 4.5 |
| | 0.008 | 5.0 | 4.0 | 1.0 | 1.0 | 1.0 | 9.0 | 2.0 | 1.0 | 5.0 | 3.0 | 3.5 | 5.0 |
| 36 | 0.062 | 9.0 | 5.0 | 0.0 | 4.0 | 3.0 | 9.0 | 3.0 | 9.0 | 4.5 | 4.0 | 7.0 | 8.5 |
| | 0.032 | 9.0 | 5.0 | 0.0 | 2.0 | 2.0 | 9.0 | 0.0 | 7.0 | 4.0 | 2.5 | 6.0 | 8.0 |
| | 0.016 | 7.0 | 4.0 | 0.0 | 0.0 | 0.0 | 8.0 | 0.0 | 4.0 | 4.0 | 1.0 | 5.0 | 7.5 |
| | 0.008 | 2.0 | 2.0 | 0.0 | 0.0 | 0.0 | 5.0 | 0.0 | 4.0 | 3.5 | 1.5 | 4.5 | 7.5 |
| 37 | 0.062 | 9.0 | 9.0 | 0.0 | 4.0 | 2.0 | 9.0 | 3.0 | 9.0 | 7.0 | 7.5 | 7.0 | 8.0 |
| | 0.032 | 5.0 | 9.0 | 0.0 | 4.0 | 0.0 | 9.0 | 2.0 | 7.0 | 6.5 | 3.5 | 5.5 | 7.5 |
| | 0.016 | 3.0 | 4.0 | 0.0 | 0.0 | 0.0 | 9.0 | 0.0 | 0.0 | 4.0 | 1.0 | 5.5 | 7.5 |
| | 0.008 | 0.0 | 0.0 | 0.0 | 0.0 | 0.0 | 3.0 | 0.0 | 0.0 | 3.5 | 0.5 | 3.5 | 7.0 |
| 38 | 0.062 | 9.0 | 9.0 | 1.0 | 3.0 | 4.0 | 9.0 | 3.0 | 9.0 | 7.5 | 6.0 | 8.0 | 8.0 |
| | 0.032 | 9.0 | 7.0 | 0.0 | 2.0 | 3.0 | 9.0 | 3.0 | 7.0 | 6.0 | 3.5 | 6.0 | 8.0 |
| | 0.016 | 8.0 | 7.0 | 0.0 | 0.0 | 0.0 | 5.0 | 0.0 | 0.0 | 5.5 | 2.0 | 4.5 | 8.0 |
| | 0.008 | 3.0 | 2.0 | 0.0 | 0.0 | 0.0 | 5.0 | 0.0 | 0.0 | 5.5 | 1.0 | 3.5 | 7.5 |
| 39 | 0.062 | 9.0 | 7.0 | 2.0 | 7.0 | 3.0 | 9.0 | 3.0 | 2.0 | 5.0 | 4.0 | 5.5 | 7.5 |
| | 0.032 | 9.0 | 6.0 | 0.0 | 2.0 | 3.0 | 9.0 | 0.0 | 0.0 | 4.0 | 1.0 | 3.5 | 7.5 |
| | 0.016 | 9.0 | 0.0 | 0.0 | 2.0 | 0.0 | 8.0 | 0.0 | 0.0 | 3.5 | 0.5 | 3.0 | 7.5 |
| | 0.008 | 9.0 | 0.0 | 0.0 | 0.0 | 0.0 | 9.0 | 0.0 | 0.0 | 3.5 | 0.0 | 2.5 | 7.0 |
| 40 | 0.062 | 9.0 | 7.0 | 6.0 | 9.0 | 5.0 | 9.0 | 9.0 | 9.0 | 7.0 | 6.5 | 6.5 | 8.5 |
| | 0.032 | 9.0 | 9.0 | 3.0 | 7.0 | 2.0 | 9.0 | 9.0 | 9.0 | 6.5 | 5.0 | 5.5 | 7.5 |
| | 0.016 | 9.0 | 7.0 | 1.0 | 3.0 | 2.0 | 9.0 | 9.0 | 9.0 | 6.0 | 5.0 | 5.0 | 7.5 |
| | 0.008 | 9.0 | 9.0 | 1.0 | 4.0 | 1.0 | 9.0 | 4.0 | 6.0 | 5.5 | 4.0 | 4.0 | 5.0 |
| 41 | 0.062 | 9.0 | 9.0 | 9.0 | 5.0 | 3.0 | 9.0 | 9.0 | 9.0 | 8.0 | 8.0 | 8.0 | 9.0 |
| | 0.032 | 9.0 | 9.0 | 4.0 | 4.0 | 3.0 | 9.0 | 9.0 | 9.0 | 8.0 | 7.0 | 8.0 | 8.0 |
| | 0.016 | 9.0 | 9.0 | 3.0 | 2.0 | 1.0 | 9.0 | 9.0 | 9.0 | 7.5 | 2.5 | 7.0 | 8.0 |
| | 0.008 | 9.0 | 7.0 | 1.0 | 2.0 | 0.0 | 9.0 | 6. | 7.0 | 5.5 | 1.5 | 6.0 | 6.5 |
| 42 | 0.062 | 9.0 | 9.0 | 4.0 | 7.0 | 1.0 | 9.0 | 1.0 | 9.0 | 7.0 | 2.5 | 4.0 | 5.0 |
| | 0.032 | 9.0 | 7.0 | 4.0 | 7.0 | 1.0 | 9.0 | 1.0 | 9.0 | 7.0 | 2.5 | 3.0 | 4.5 |
| | 0.016 | 9.0 | 7.0 | 0.0 | 6.0 | 1.0 | 9.0 | 1.0 | 2.0 | 5.5 | 2.5 | 2.5 | 5.0 |
| | 0.008 | 7.0 | 7.0 | 0.0 | 2.0 | 1.0 | 9.0 | 0.0 | 0.0 | 5.0 | 2.0 | 2.5 | 5.0 |
| 43 | 0.062 | 9.0 | 9.0 | 3.0 | 4.0 | 1.0 | 9.0 | 2.0 | 9.0 | 5.5 | 3.0 | 3.0 | 5.0 |
| | 0.032 | 9.0 | 7.0 | 0.0 | 3.0 | 1.0 | 9.0 | 1.0 | 9.0 | 5.5 | 2.0 | 3.0 | 5.0 |
| | 0.016 | 9.0 | 7.0 | 0.0 | 3.0 | 0.0 | 9.0 | 1.0 | 1.0 | 5.0 | 2.0 | 3.0 | 5.0 |
| | 0.008 | 9.0 | 1.0 | 0.0 | 0.0 | 0.0 | 9.0 | 1.0 | 0.0 | 5.0 | 2.5 | 3.0 | 4.0 |
| 44 | 0.062 | 9.0 | 7.0 | 2.0 | 9.0 | 2.0 | 9.0 | 6.0 | 7.0 | 7.0 | 3.0 | 4.0 | 7.0 |
| | 0.032 | 7.0 | 6.0 | 1.0 | 6.0 | 1.0 | 9.0 | 6.0 | 7.0 | 6.5 | 2.5 | 3.5 | 6.5 |
| | 0.016 | 9.0 | 3.0 | 0.0 | 7.0 | 0.0 | 7.0 | 4.0 | 4.0 | 6.5 | 2.5 | 4.0 | 6.5 |
| | 0.008 | 9.0 | 3.0 | 0.0 | 5.0 | 0.0 | 7.0 | 3.0 | 4.0 | 5.0 | 2.5 | 3.0 | 4.5 |
| 45 | 0.062 | 9.0 | 9.0 | 5.0 | 7.0 | 3.0 | 9.0 | 9.0 | 9.0 | 8.0 | 3.5 | 8.0 | 9.0 |
| | 0.032 | 9.0 | 7.0 | 4.0 | 7.0 | 1.0 | 9.0 | 8.0 | 9.0 | 8.0 | 2.0 | 6.0 | 8.0 |
| | 0.016 | 9.0 | 3.0 | 0.0 | 4.0 | 0.0 | 9.0 | 5.0 | 8.0 | 6.5 | 1.0 | 6.0 | 8.0 |
| | 0.008 | 9.0 | 2.0 | 0.0 | 2.0 | 0.0 | 9.0 | 0.0 | 3.0 | 6.0 | 0.5 | 4.0 | 7.5 |

### EXAMPLE 47

### Preemergence Herbicidal Evaluation of Test Compounds

The preemergence herbicidal activity of the test compounds of the present invention is exemplified by the following tests in which the seeds of a variety of monocotyledonous and dicotyledonous plants are separately mixed with potting soil and planted on top of approximately one inch of soil in separate pint cups. After planting, the cups are sprayed with the selected aqueous acetone solution containing test compound in sufficient quantity to provide the equivalent of about 0.013 to 0.800 kg per hectare of test compound per cup. The treated cups are then placed on greenhouse benches, watered and cared for in accordance with conventional greenhouse procedures. From four to five weeks after treatment, the tests are terminated and each cup is examined and rated according to the rating system provided in Example 46. The data obtained are reported in Table II below.

TABLE II
PREEMERGENCE HERBICIDAL EVALUATION

| Ex. | Rate | | | | | | | | | | | | |
|---|---|---|---|---|---|---|---|---|---|---|---|---|---|
| No. | (kg/ha) | ABUTH | AMBEL | CASOB | CHEAL | GALAP | IPOHE | ECHCG | SETVI | GLXMAW | ORYSAT | TRZAWR | ZEAMX |
| 16 | 0.125 | 9.0 | 9.0 | 9.0 | 9.0 | 9.0 | 9.0 | 9.0 | 9.0 | 9.0 | 9.0 | 9.0 | 8.0 |
| | 0.062 | 9.0 | 9.0 | 9.0 | 9.0 | 9.0 | 9.0 | 9.0 | 9.0 | 9.0 | 9.0 | 8.0 | 8.0 |
| | 0.032 | 9.0 | 9.0 | 0.0 | 9.0 | 0.0 | 3.0 | 9.0 | 9.0 | 9.0 | 7.0 | 8.0 | 7.0 |
| | 0.016 | 9.0 | 6.0 | 0.0 | 9.0 | 0.0 | 3.0 | 9.0 | 9.0 | 9.0 | 7.0 | 6.0 | 8.0 |
| 17 | 0.125 | 9.0 | 8.3 | 2.3 | 9.0 | 1.0 | 9.0 | 8.3 | 9.0 | 3.0 | 4.7 | | 6.7 |
| | 0.062 | 9.0 | 6.7 | 3.3 | 9.0 | 1.0 | 6.0 | 6.3 | 9.0 | 3.0 | 4.0 | 3.0 | 4.0 |
| | 0.032 | 7.0 | 3.3 | 1.3 | 9.0 | 0.0 | 3.3 | 1.7 | 7.3 | 1.7 | 0.5 | | 3.7 |
| | 0.016 | 9.0 | 0.0 | 0.0 | 9.0 | 0.0 | 0.0 | 0.0 | 6.5 | 1.5 | 1.0 | | 1.0 |
| 18 | 0.125 | 9.0 | 8.0 | 5.0 | 9.0 | 9.0 | 9.0 | 9.0 | 9.0 | 9.0 | 6.0 | 7.0 | 8.5 |
| | 0.062 | 9.0 | 9.0 | 0.5 | 9.0 | 5.0 | 8.0 | 9.0 | 9.0 | 7.5 | 6.0 | 6.0 | 7.0 |
| | 0.032 | 9.0 | 6.0 | 0.0 | 9.0 | 1.0 | 9.0 | 8.0 | 9.0 | 4.5 | 6.0 | 3.0 | 5.0 |
| | 0.016 | 6.0 | 4.0 | 2.5 | 9.0 | | 7.5 | 6.0 | 9.0 | 3.0 | 3.0 | 2.0 | 5.0 |
| 19 | 0.125 | 9.0 | 9.0 | 1.0 | 9.0 | 9.0 | 8.0 | 9.0 | 9.0 | 4.0 | 2.0 | 3.0 | 6.5 |
| | 0.062 | 9.0 | 8.0 | 1.5 | 9.0 | 0.0 | 5.5 | 7.0 | 9.0 | 2.5 | 1.5 | 2.0 | 4.0 |
| | 0.032 | 6.5 | 5.5 | 1.5 | 9.0 | 0.0 | 1.5 | 4.0 | 7.0 | 1.5 | 0.5 | 1.0 | 1.5 |
| | 0.016 | 1.0 | 4.5 | 1.0 | 9.0 | 0.0 | 1.0 | 0.5 | 4.5 | 0.5 | 0.0 | 0.0 | 1.0 |
| 20 | 0.125 | 9.0 | 5.5 | 2.0 | 9.0 | 0.0 | 4.5 | 8.0 | 9.0 | 3.0 | 3.0 | 6.0 | 6.0 |
| | 0.062 | 9.0 | 2.5 | 0.0 | 9.0 | 0.0 | 4.5 | 3.5 | 9.0 | 1.5 | 1.0 | 4.0 | 4.5 |
| | 0.032 | 6.5 | 1.5 | 0.0 | 9.0 | 0.0 | 3.0 | 0.0 | 8.0 | 0.5 | 0.0 | 0.0 | 1.0 |
| | 0.016 | 0.0 | 0.0 | 0.0 | 9.0 | 0.0 | 0.0 | 0.0 | 0.0 | 0.0 | 0.0 | 0.0 | 0.0 |
| 21 | 0.125 | 9.0 | 7.5 | 2.0 | 9.0 | 0.0 | 3.5 | 3.5 | 9.0 | 1.0 | 0.0 | 1.0 | 3.5 |
| | 0.062 | 6.0 | 3.5 | 1.5 | 9.0 | 0.0 | 2.0 | 1.0 | 6.5 | 0.5 | 0.0 | 0.0 | 1.0 |
| | 0.032 | 4.5 | 3.0 | 0.0 | 9.0 | 0.0 | 1.5 | 0.0 | 2.0 | 0.0 | 0.0 | 0.0 | 0.0 |
| | 0.016 | 0.0 | 0.0 | 0.0 | 9.0 | 0.0 | 0.0 | 0.0 | 0.0 | 0.0 | 0.0 | 0.0 | 0.0 |
| 22 | 0.125 | 7.0 | 0.0 | 0.0 | 9.0 | 0.0 | 0.0 | 7.0 | 9.0 | 0.0 | 0.0 | 4.0 | 6.0 |
| | 0.062 | 9.0 | 0.0 | 0.0 | 9.0 | 0.0 | 0.0 | 2.0 | 7.0 | 0.0 | 0.0 | 2.0 | 4.0 |
| | 0.032 | 9.0 | 0.0 | 0.0 | 9.0 | 0.0 | 0.0 | 0.0 | 2.0 | 0.0 | 0.0 | 1.0 | 2.0 |
| | 0.016 | 0.0 | 0.0 | 0.0 | 9.0 | 0.0 | 0.0 | 0.0 | 0.0 | 0.0 | 0.0 | 0.0 | 0.0 |
| 23 | 0.125 | 9.0 | 1.0 | 0.0 | 9.0 | 0.0 | 1.0 | 8.0 | 9.0 | 0.0 | 0.0 | 3.0 | 4.0 |
| | 0.062 | 9.0 | 0.0 | 0.0 | 9.0 | 0.0 | 0.0 | 5.0 | 9.0 | 0.0 | 0.0 | 1.0 | 0.0 |
| | 0.032 | 3.0 | 0.0 | 0.0 | 7.0 | 0.0 | 0.0 | 2.0 | 8.0 | 0.0 | 0.0 | 0.0 | 0.0 |
| | 0.016 | 0.0 | 0.0 | 0.0 | 0.0 | 0.0 | 0.0 | 0.0 | 3.0 | 0.0 | 0.0 | 0.0 | 0.0 |
| 24 | 0.125 | 9.0 | 0.0 | 0.0 | 9.0 | | 0.0 | 7.0 | 9.0 | 5.0 | 1.0 | 1.0 | 6.0 |
| | 0.062 | 9.0 | 0.0 | 0.0 | 9.0 | 0.0 | 0.0 | 3.0 | 9.0 | 2.0 | 1.0 | 1.0 | 4.0 |
| | 0.032 | 6.0 | 0.0 | 0.0 | 0.0 | 0.0 | 0.0 | 0.0 | 0.0 | 1.0 | 0.0 | 0.0 | 0.0 |
| | 0.016 | 0.0 | 0.0 | 0.0 | 0.0 | 0.0 | 0.0 | 0.0 | 0.0 | 0.0 | 0.0 | 0.0 | 0.0 |
| 25 | 0.125 | 9.0 | 3.5 | 2.0 | 9.0 | 0.0 | 6.5 | 8.0 | 9.0 | 1.5 | 1.5 | 2.0 | 4.0 |
| | 0.062 | 6.0 | 1.0 | 1.5 | 9.0 | 0.0 | 5.5 | 2.0 | 9.0 | 2.0 | 0.0 | 0.0 | 2.0 |
| | 0.032 | 6.0 | 0.0 | 0.0 | 9.0 | 0.0 | 4.5 | 0.0 | 3.5 | 0.0 | 0.0 | 0.0 | 0.0 |
| | 0.016 | 3.0 | 0.0 | 0.0 | 9.0 | 0.0 | 0.0 | 0.0 | 0.0 | 0.0 | 0.0 | 0.0 | 0.0 |
| 26 | 0.125 | 9.0 | 9.0 | 7.0 | 9.0 | 8.0 | 9.0 | 9.0 | 9.0 | 5.0 | 7.0 | 8.0 | 7.0 |
| | 0.062 | 9.0 | 7.0 | 7.0 | 9.0 | 7.0 | 6.0 | 7.0 | 9.0 | 9.0 | 5.0 | 6.0 | 7.0 |
| | 0.032 | 9.0 | 7.0 | 0.0 | 7.0 | 0.0 | 9.0 | 6.0 | 9.0 | 2.0 | 3.0 | | 4.0 |
| | 0.016 | 9.0 | 2.0 | 0.0 | | 0.0 | 0.0 | 1.0 | 2.0 | 1.0 | 0.0 | 0.0 | 1.0 |
| 27 | 0.125 | 9.0 | 9.0 | 9.0 | 9.0 | 9.0 | 5.0 | 9.0 | 9.0 | 9.0 | 6.0 | 2.0 | 7.0 |
| | 0.062 | 9.0 | 7.0 | 2.0 | 9.0 | 9.0 | 1.0 | 9.0 | 9.0 | 5.0 | 5.0 | 3.0 | 7.0 |
| | 0.032 | 6.0 | 2.0 | 1.0 | 9.0 | 2.0 | 1.0 | 4.0 | 9.0 | 1.0 | 2.0 | 2.0 | 4.0 |
| | 0.016 | | 0.0 | 0.0 | 9.0 | 1.0 | 0.0 | 1.0 | 9.0 | 1.0 | 1.0 | 1.0 | 2.0 |
| 28 | 0.125 | 9.0 | 7.0 | | 9.0 | 9.0 | 1.0 | 7.0 | 9.0 | 0.0 | 0.0 | 0.0 | 5.0 |
| | 0.062 | 9.0 | 1.0 | 0.0 | 9.0 | 0.0 | 1.0 | 0.0 | 4.0 | 0.0 | 0.0 | 0.0 | 4.0 |
| | 0.032 | 2.0 | 0.0 | 0.0 | 9.0 | 0.0 | 0.0 | 0.0 | 2.0 | 0.0 | 0.0 | 0.0 | 3.0 |
| | 0.016 | | 0.0 | 0.0 | 3.0 | 0.0 | 0.0 | 0.0 | 0.0 | 0.0 | 0.0 | 0.0 | 0.0 |
| 29 | 0.125 | 9.0 | 7.0 | 1.0 | 9.0 | 8.0 | 7.0 | 7.0 | 9.0 | 5.0 | 4.0 | 2.0 | 5.0 |
| | 0.062 | 9.0 | 3.0 | | 9.0 | 1.0 | 6.0 | 7.0 | 9.0 | 4.0 | 3.0 | 2.0 | 4.0 |
| | 0.032 | 9.0 | 0.0 | | 9.0 | 0.0 | 3.0 | 4.0 | 9.0 | 1.0 | 1.0 | 1.0 | 2.0 |
| | 0.016 | 6.0 | 0.0 | 1.0 | 9.0 | 0.0 | 0.0 | 2.0 | 6.0 | 0.0 | 0.0 | 1.0 | 0.0 |
| 30 | 0.125 | 6.0 | 6.0 | 2.0 | 9.0 | 4.0 | 3.0 | 7.0 | 9.0 | 2.0 | 4.0 | 2.0 | 7.0 |
| | 0.062 | | 3.0 | 0.0 | 9.0 | 3.0 | 4.0 | 7.0 | 9.0 | 1.0 | 2.0 | 2.0 | 5.0 |
| | 0.032 | | 0.0 | 1.0 | 9.0 | 2.0 | 0.0 | 5.0 | 7.0 | 1.0 | 1.0 | 1.0 | 3.0 |
| | 0.016 | 0.0 | 0.0 | 0.0 | 9.0 | 2.0 | 0.0 | 1.0 | 4.0 | 1.0 | 0.0 | 0.0 | 1.0 |
| 31 | 0.125 | 3.0 | 0.0 | 0.0 | 9.0 | 0.0 | 0.0 | 0.0 | 0.0 | 0.0 | 0.0 | 0.0 | 0.0 |
| | 0.062 | 0.0 | 0.0 | 0.0 | 3.0 | 0.0 | 0.0 | 0.0 | 0.0 | 0.0 | 0.0 | 0.0 | 0.0 |
| | 0.032 | 0.0 | 0.0 | 0.0 | 0.0 | 0.0 | 0.0 | 0.0 | 0.0 | 0.0 | 0.0 | 0.0 | 0.0 |
| | 0.016 | 0.0 | 0.0 | 0.0 | 0.0 | 0.0 | 0.0 | 0.0 | 0.0 | 0.0 | 0.0 | 0.0 | 0.0 |
| | 0.008 | 0.0 | 0.0 | 0.0 | 0.0 | 0.0 | 0.0 | 0.0 | 0.0 | 0.0 | 0.0 | 0.0 | 0.0 |
| 32 | 0.125 | 4.0 | 0.0 | 0.0 | 6.0 | | 0.0 | 0.0 | 0.0 | 1.0 | 0.0 | 0.0 | 1.0 |
| | 0.062 | 0.0 | 0.0 | 0.0 | 0.0 | 0.0 | 0.0 | 0.0 | 0.0 | 0.0 | 0.0 | 0.0 | 0.0 |
| | 0.032 | 0.0 | 0.0 | 0.0 | 0.0 | 0.0 | 0.0 | 0.0 | 0.0 | 0.0 | 0.0 | 0.0 | 0.0 |
| | 0.016 | 0.0 | 0.0 | 0.0 | 0.0 | 0.0 | 0.0 | 0.0 | 0.0 | 0.0 | 0.0 | 0.0 | 0.0 |
| 33 | 0.125 | 9.0 | 0.0 | 0.0 | 9.0 | 0.0 | 0.0 | 0.0 | 0.0 | 0.0 | 0.0 | 0.0 | 0.0 |
| | 0.062 | 0.0 | 0.0 | 0.0 | 9.0 | 0.0 | 0.0 | 0.0 | 0.0 | 0.0 | 0.0 | 0.0 | 0.0 |
| | 0.032 | 0.0 | 0.0 | 0.0 | 9.0 | 0.0 | 0.0 | 0.0 | 0.0 | 0.0 | 0.0 | 0.0 | 0.0 |
| | 0.016 | 0.0 | 0.0 | 0.0 | 9.0 | 0.0 | 0.0 | 0.0 | 0.0 | 0.0 | 0.0 | 0.0 | 0.0 |
| 34 | 0.125 | 9.0 | 8.0 | 6.0 | 9.0 | 7.0 | 9.0 | 7.5 | 9.0 | 2.0 | 5.0 | 3.0 | 4.0 |
| | 0.062 | 9.0 | 6.0 | 2.5 | 9.0 | 0.0 | 5.0 | 5.0 | 9.0 | 1.5 | 3.0 | 1.0 | 3.5 |
| | 0.032 | 9.0 | 5.0 | 2.0 | 9.0 | 0.0 | 9.0 | 2.0 | 6.0 | 1.0 | 2.5 | 1.0 | 0.5 |
| | 0.016 | 9.0 | 1.0 | | 9.0 | 0.0 | | 4.0 | 7.0 | 1.0 | 1.0 | 1.0 | 0.0 |
| 35 | 0.125 | 5.5 | 5.0 | 2.0 | 9.0 | 5.0 | 9.0 | 4.5 | 9.0 | 2.5 | 6.0 | 1.0 | 3.0 |
| | 0.062 | 9.0 | 3.5 | 4.0 | 9.0 | 0.0 | 5.0 | 2.0 | 6.5 | 1.5 | 3.5 | 1.0 | 1.0 |
| | 0.032 | 4.0 | 0.0 | 0.0 | 9.0 | 0.0 | 4.5 | 0.0 | 1.5 | 0.0 | 0.0 | 0.0 | 0.0 |
| | 0.016 | 0.0 | 0.0 | 0.0 | 0.0 | 0.0 | 0.0 | 0.0 | 0.0 | 1.0 | 0.0 | 0.0 | 0.0 |
| 36 | 0.125 | 8.0 | 5.0 | 0.0 | 9.0 | 0.0 | 4.0 | 0.0 | 3.0 | 0.0 | 1.5 | 0.0 | 0.0 |
| | 0.062 | 3.0 | 2.0 | 0.0 | 9.0 | 0.0 | 0.0 | 0.0 | 0.0 | 0.0 | 1.0 | 0.0 | 0.0 |
| | 0.032 | 1.5 | 2.0 | 0.0 | 6.5 | 0.0 | 0.0 | 0.0 | 0.0 | 0.0 | 0.0 | 0.0 | 0.0 |
| | 0.016 | 0.0 | 0.0 | 0.0 | 0.0 | 0.0 | 0.0 | 0.0 | 0.0 | 0.0 | 0.0 | 0.0 | 0.0 |
| 37 | 0.125 | 9.0 | 3.0 | 0.0 | 9.0 | 0.0 | 5.0 | 0.0 | 8.0 | 0.0 | 0.0 | 0.0 | 0.0 |
| | 0.062 | 9.0 | 0.0 | 0.0 | 0.0 | 0.0 | 0.0 | 0.0 | 0.0 | 0.0 | 0.0 | 0.0 | 0.0 |
| | 0.032 | 0.0 | 0.0 | 0.0 | | 0.0 | 0.0 | 0.0 | 0.0 | 0.0 | 0.0 | 0.0 | 0.0 |
| | 0.016 | 0.0 | 0.0 | 0.0 | | 0.0 | 0.0 | 0.0 | 0.0 | 0.0 | 0.0 | 0.0 | 0.0 |
| 38 | 0.125 | 9.0 | 9.0 | 2.0 | 9.0 | 0.0 | | 0.0 | 9.0 | 0.0 | 0.0 | 0.0 | 0.0 |
| | 0.062 | 2.0 | 0.0 | 0.0 | 9.0 | 0.0 | 0.0 | 0.0 | 0.0 | 0.0 | 0.0 | 0.0 | 0.0 |
| | 0.032 | 0.0 | 0.0 | 0.0 | 9.0 | 0.0 | 0.0 | 0.0 | 0.0 | 0.0 | 0.0 | 0.0 | 0.0 |
| | 0.016 | 0.0 | 0.0 | 0.0 | 9.0 | 0.0 | 0.0 | 0.0 | 0.0 | 0.0 | 0.0 | 0.0 | 0.0 |
| 39 | 0.125 | 9.0 | 0.0 | 0.0 | 9.0 | 7.0 | 3.0 | 0.0 | 4.0 | 0.0 | 3.0 | 0.0 | 0.0 |
| | 0.062 | 9.0 | 0.0 | 0.0 | 9.0 | 0.0 | 0.0 | 0.0 | 0.0 | 0.0 | 0.0 | 0.0 | 0.0 |
| | 0.032 | 5.0 | 0.0 | 0.0 | 3.0 | 0.0 | 0.0 | 0.0 | 0.0 | 0.0 | 0.0 | 0.0 | 0.0 |
| | 0.016 | 0.0 | 0.0 | 0.0 | 3.0 | 0.0 | 0.0 | 0.0 | 0.0 | 0.0 | 0.0 | 0.0 | 0.0 |
| 40 | 0.125 | 9.0 | 4.0 | 4.0 | 9.0 | 9.0 | 9.0 | 4.0 | 6.0 | 0.0 | 2.0 | 2.0 | 1.0 |
| | 0.062 | 9.0 | 3.0 | 3.0 | 9.0 | 7.0 | 3.0 | 0.0 | 5.0 | 1.0 | 0.0 | 0.0 | 1.0 |
| | 0.032 | 9.0 | 4.0 | 4.0 | 9.0 | 0.0 | 1.0 | 0.0 | 4.0 | 0.0 | 0.0 | 0.0 | 0.0 |
| | 0.016 | 1.0 | 0.0 | 0.0 | 9.0 | | 0.0 | 0.0 | 0.0 | 0.0 | 0.0 | 0.0 | 0.0 |
| 41 | 0.125 | 9.0 | 7.0 | | 9.0 | 0.0 | 9.0 | 4.0 | 9.0 | 2.0 | 4.0 | 1.0 | 0.0 |
| | 0.062 | 9.0 | 0.0 | 0.0 | 9.0 | 0.0 | 0.0 | 0.0 | 0.0 | 2.0 | 3.0 | 0.0 | 0.0 |
| | 0.032 | 4.0 | 0.0 | 0.0 | 9.0 | 0.0 | 0.0 | 0.0 | 0.0 | 0.0 | 0.0 | 0.0 | 0.0 |
| | 0.016 | 4.0 | 0.0 | 0.0 | 4.0 | 0.0 | 0.0 | 0.0 | 0.0 | 0.0 | 0.0 | 0.0 | 0.0 |
| 42 | 0.125 | 9.0 | 5.0 | 1.0 | 9.0 | 0.0 | 6.0 | 7.0 | 9.0 | 5.0 | 1.0 | 1.0 | 4.0 |
| | 0.062 | 9.0 | 1.0 | 0.0 | 9.0 | 0.0 | 0.0 | 3.0 | 9.0 | 3.0 | 1.0 | 0.0 | 1.0 |
| | 0.032 | 9.0 | 0.0 | 0.0 | 9.0 | 0.0 | 0.0 | 3.0 | 7.0 | 3.0 | 0.0 | 0.0 | 0.0 |
| | 0.016 | 7.0 | 0.0 | 0.0 | 9.0 | 0.0 | 0.0 | 1.0 | 3.0 | 0.0 | 0.0 | 0.0 | 0.0 |
| 43 | 0.125 | 8.0 | 0.0 | 0.0 | 9.0 | 0.0 | 0.0 | 1.0 | 7.0 | 1.0 | 0.0 | 1.0 | 2.0 |
| | 0.062 | 9.0 | 0.0 | 0.0 | 9.0 | 0.0 | 0.0 | 0.0 | 4.0 | 0.0 | 0.0 | 0.0 | 0.0 |
| | 0.032 | 1.0 | 0.0 | 0.0 | 9.0 | 0.0 | 0.0 | 0.0 | 1.0 | 1.0 | 0.0 | 0.0 | 0.0 |
| | 0.016 | 0.0 | 0.0 | 0.0 | 9.0 | 0.0 | 0.0 | 0.0 | 0.0 | 1.0 | 0.0 | 0.0 | 0.0 |
| 44 | 0.125 | 9.0 | 0.0 | 0.0 | 9.0 | 7.0 | 2.0 | 0.0 | 9.0 | 0.0 | 5.0 | 1.0 | 1.0 |
| | 0.062 | 7.0 | 0.0 | 2.0 | 9.0 | 4.0 | 0.0 | 0.0 | 3.0 | 0.0 | 5.0 | 1.0 | 1.0 |
| | 0.032 | 0.0 | 0.0 | 1.0 | 9.0 | 5.0 | 0.0 | 0.0 | 0.0 | 0.0 | 0.0 | 0.0 | 0.0 |
| | 0.016 | | 1.0 | 0.0 | 9.0 | 3.0 | 0.0 | 0.0 | 0.0 | 0.0 | 0.0 | 0.0 | 0.0 |
| 45 | 0.125 | 9.0 | 0.0 | | 9.0 | 4.0 | 4.0 | 0.0 | 9.0 | 1.0 | 5.0 | 1.0 | 0.0 |
| | 0.062 | 4.0 | 0.0 | 0.0 | 9.0 | 4.0 | 0.0 | 0.0 | 9.0 | 0.0 | 0.0 | 0.0 | 0.0 |
| | 0.032 | 0.0 | 0.0 | 0.0 | 0.0 | 0.0 | 0.0 | 0.0 | 0.0 | 0.0 | 0.0 | 0.0 | 0.0 |
| | 0.016 | 0.0 | 0.0 | 0.0 | 0.0 | 0.0 | 0.0 | 0.0 | 0.0 | 0.0 | 0.0 | 0.0 | 0.0 |

what is claimed is:

1. A compound of formula I 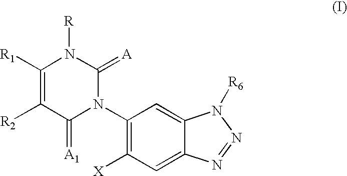
wherein
R is C₁-C₆alkyl, C₁-C₆haloalkyl, C₃-C₇cycloalkyl, C₃-C₆alkenyl, C₃-C₆alkynyl, benzyl, OR₃ or NR₄R₅;
R₁ and R₂ are each independently hydrogen, halogen, C₁-C6alkyl, C₁-C₆haloalkyl, C₃-C₇cycloalkyl, C₃-C₆alkenyl or C₁-C₆alkynyl;
R₃, R₄ and R₅ are each independently hydrogen, C₁-C₆alkyl, C₁-C₆haloalkyl, C₂-C₆alkoxyalkyl, C₃-C₇cycloalkyl, C₃-C₆alkenyl, C₃-C₆alkynyl or benzyl;
X is hydrogen or halogen;
A and A₁ are each independently O or S;
R₆ is phenyl optionally substituted with any combination of one to three halogen atoms, one to three C₁-C₈ alkyl groups, one to three C₁-C₆alkoxy groups, one C₁-C₄alkoxy-C₁-C₄alkyl group, one C₃-C₇cycloalkyl group, one or two C₁-C₆haloalkyl groups, one C₁-C₄alkylthio group, one cyano group, one nitro group, one C(O)R₇ group, one CO₂R₈ group, one or two X₄R₉ groups, one CH=CHR₁₀ group, one CH₂CH(R₁₁)R₁₂ group or one N(R₁₃)SO₂R₁₄ group;
R₇, R₁₃, R₁₄, R₁₆, R₁₇, R₁₈, R₂₂, R₂₃, R₂₆, R₂₇, R₂₉ and R₃₀ are each independently hydrogen, C₁-C₆alkyl, C₁-C₆haloalkyl, C₃-C₇cycloalkyl, C₃-C₆alkenyl, C₃-C₆alkynyl, benzyl or phenyl;
R₈, R₂₁, R₂₅ and R₂₈ are each independently hydrogen, C₁-C₁₀alkyl, C₁-C₈haloalkyl, C₃-C₇cycloalkyl, C₃-C₇halocycloalkyl, C₃-C₁₀ alkenyl, C₃-C₈haloalkenyl, C₅-C₈cycloalkenyl, C₅-C₈halocycloalkenyl, C₃-C₈alkynyl, C₃-C₈haloalkynyl, benzyl, phenyl, furfuryl, pyridyl, thienyl, oximino, or an alkali metal, alkaline earth metal, manganese, copper, zinc, cobalt, silver, nickel, ammonium or organic ammonium cation;
R₉ is hydrogen, Si(R₁₅)₃, C(O)NR₁₆R₁₇, C(O)R₁₈, SO₂R₁₉, or C₁-C₁₀alkyl, C₃-C₁₀alkenyl, C₅-C₈cycloalkenyl, C₃-C₈cycloalkyl, C₃-C₁₀alkynyl, phenyl or benzyl, each of which may optionally be substituted with one to three halogen atoms, one hydroxyl, one benzyloxy, one C₁-C₆ alkoxy group, one phenoxy group, one OC(O)R₂₀ group, one CO2R21 group, one C(O)R₂₂ group, one C(O)R₂₃R₂₄ group or one cyano group;
R₁₀ and R₁₂ are each independently hydrogen, C₁-C₆alkyl, CO₂R₂₅, C(O)R₂₆ or C(O)NR₂₇R₂₈;
R₁₁ is hydrogen, halogen, C₁-C₆alkyl, C₁-C₆haloalkyl, C₃-C₇cycloalkyl, C₃-C₆alkenyl, C₃-C₆alkynyl, benzyl, phenyl or cyano;
R₁₉ is C₁-C₆alkyl, C₁-C₆haloalkyl, C₃-C₇cycloalkyl, C₃-C₆alkenyl, C₃-C₆alkynyl, benzyl, phenyl or cyano;
R₂₄ is hydrogen, C₁-C₆alkyl, C₁-C₆alkoxy, C₁-C₆haloalkyl, C₃-C₇cycloalkyl, C₃-C₆alkenyl, C₃-C₆alkynyl, SO₂R₂₉, C(O)R_{30,} benzyl or phenyl;
R₁₅ and R₂₀ are each independently C₁-C₆alkyl, C₃-C₆alkenyl, C₃-C₆alkynyl, benzyl or phenyl; and
X₄ is O, S or NH.

2. The compound according to claim 1 wherein
R is C₁-C₃alkyl or amino;
R₁ is C₁- C₃haloalkyl;
R₂ is hydrogen;
X is halogen;
A and A₁ are O;
R₆ is phenyl optionally substituted with any combination of one to three halogen atoms, one to three C₁-C₈alkyl groups, one to three C₁-C₆alkoxy groups, one methoxy-methyl group, one C₃-C₇cycloalkyl group, one or two C₁-C₆haloalkyl groups, one C₁-C₄alkylthio group, one cyano group, one nitro group, one C(O)R₇ group, one CO₂R₈ group, one or two X₄R₉, groups, one CH=CHR₁₀ group, one CH₂CH(R₁₁) R₁₂ group or one N (R₁₃) SO₂R₁₄ group;
R₇, R₁₃, R₁₄, R₁₆, R₁₇, R₁₈, R₂₂, R₂₃, R₂₆, R₂₇, R₂₉ and R₃₀ are each independently hydrogen, C₁-C₆alkyl, C₁-C₆haloalkyl, C₃-C₇cycloalkyl, C₃-C₆alkenyl, C₃-C₆alkynyl, benzyl or phenyl;
R₈, R₂₁, R₂₅ and R₂₈ are each independently hydrogen, C₁-C₁₀alkyl, C₁-C₈haloalkyl, C₃-C₇cycloalkyl, C₃-C₇halocycloalkyl, C₃-C₁₀ alkenyl, C₃-C₈haloalkenyl, C₅-C₈Cycloalkenyl, C₅-C₈halocycloalkenyl, C₃-C₈alkynyl, C₃-C₈haloalkynyl, benzyl, phenyl, furfuryl, pyridyl, thienyl, oximino, or an alkali metal, alkaline earth metal, manganese, copper, zinc, cobalt, silver, nickel, ammonium or organic ammonium cation;
R₉ is hydrogen, Si(R₁₅)₃, C(O)NR₁₆R₁₇, C(O)R₁₈, SO₂R₁₉, or C₁-C₁₀alkyl, C₃-C₁₀alkenyl, C₅-C₈cycloalkenyl, C₃-C₈cycloalkyl, C₃-C₁₀alkynyl, phenyl or benzyl, each of which may optionally be substituted with one to three halogen atoms, one hydroxyl, one benzyloxy, one C₁-C₆ alkoxy group, one phenoxy group, one OC(O)R₂₀ group, one CO2R21 group, one C(O)R₂₂ group, one C(O)R₂₃R₂₄ group or one cyano group;
R₁₀ and R₁₂ are each independently hydrogen, C₁-C₆alkyl, CO₂R₂₅, C(O)R₂₆ or C(O)NR₂₇R₂₈;
R₁₁ is hydrogen, halogen, C₁-C₆alkyl, C₁-C₆haloalkyl, C₃-C₇cycloalkyl, C₃-C₆alkenyl, C₃-C₆alkynyl, benzyl, phenyl or cyano;
R₁₉ is C₁-C₆alkyl, C₁-C₆haloalkyl, C₃-C₇cycloalkyl, C₃-C₆alkenyl, C₃-C₆alkynyl, benzyl, phenyl or cyano;
R₂₄ is hydrogen, C₁-C₆alkyl, C₁-C₆alkoxy, C₁-C₆haloalkyl, C₃-C₇cycloalkyl, C₃-C₆alkenyl, C₃-C₆alkynyl, SO₂R₂₉, C(O)R₃₀, benzyl or phenyl;
R₁₅ and R₂₀ are each independently C₁-C₆alkyl, C₃-C₆alkenyl, C₃-C₆alkynyl, benzyl or phenyl; and
X₄ is O.

3. The compound according to claim 2 wherein
R is methyl or amino;
R₁ is trifluoromethyl;
R₂ is hydrogen;
X is fluorine;
R₆ is phenyl optionally substituted with any combination of one to three halogen atoms, one to three C₁-C₄alkyl groups, one to three C₁-C₄alkoxy groups, one C₃-C₇cycloalkyl group, or one or two X₄R₉ groups;
R₉ C₁-C₃alkyl, C₃-C₆alkenyl, C₃-C₈acycloalkyl, C₃-C₆alkynyl, each of which may optionally be substituted with one halogen atom, one C₁-C₃ alkoxy group, one OC(O)R₂₀ group, one CO₂R₂₁ group, one C(O)R₂₂ group or one C(O)NR₂₃R₂₄ group;
R₂₀ is C₁-C₄alkyl, C₃-C₆alkenyl, C₃-C₆alkynyl, benzyl or phenyl;
R₂₁ hydrogen, C₁-C₄alkyl, C₃-C₇cycloalkyl, C₃-C₆alkenyl, C₃-C₆alkynyl, benzyl, phenyl, furfuryl, pyridyl, thienyl, oximino, or an alkali metal, alkaline earth metal, manganese, copper, zinc, cobalt, silver, nickel, ammonium or organic ammonium cation;
R₂₂ R₂₃ and R₂₄ are each independently hydrogen, C₁-C₄alkyl, C₁-C₄haloalkyl, C₃-C₇cycloalkyl, C₃-C₆alkenyl, C₃-C₆alkynyl, benzyl or phenyl; and
X₄ is O.

4. The compound according to claim 3 selected from the group consisting of
3-[1-(4-ethylphenyl)-5-fluoro-1H-1,2,3-benzotriazol-6-yl]-1-mentyl-6-(trifluoromethyl)-2,4(1H,3H)-pyrimidinedione;
3-[5-fluoro-1-(2-methoxy-5-methylphenyl)-1H-1,2,3-benzotriazol-6-yl ]-1-methyl-6-(trifluoromethyl)-2,4(1H,3H)-pyrimidinedione;
methyl {2-[5-fluoro-6-(3-methyl-2,6-dioxo-4-(trifluoromethyl)-3,6-dihydro-1(2H)-pyrimidinyl)-1H-1,2,3-benzotriazol-1-yl]-4-methylphenoxy}acetate; and
3-[1-(3-ethylphenyl)-5-fluoro-1H-1,2,3-benzotriazol-6-yl]-1-methyl-6-(trifluoromethyl)-2,4(1H,3H)-pyrimidinedione.

5. A method for controlling undesirable plant species which comprises applying to the foliage of said plants or to the soil or water containing seeds or other propagating organs thereof, a herbicidally effective amount of a compound of formula I. 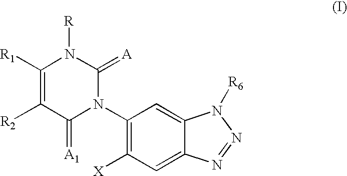
wherein
R, R₁, R₂, R₆, A, A₁ and X are defined as in claim 1.

6. The method according to claim 5 wherein
R₁ is C₁-C₃alkyl or amino;
R₁ is C₁-C₃haloalkyl;
R₂ is hydrogen;
X is halogen;
A and A₁ are O;
R₆ is phenyl optionally substituted with any combination of one to three halogen atoms, one to three C₁-C₈alkyl groups, one to three C₁-C6alkoxy groups, one C₁-C₄alkoxy-C₁-C₄alkyl group, one C₃-C₇cycloalkyl group, one or two C₁-C₆haloalkyl groups, one C₁-C₄alkylthio group, one cyano group, one nitro group, one C(O)R₇ group, one CO₂R₈ group, one or two X₄R₉, groups, one CH=CHR₁₀ group, one CH₂CH(R₁₁)R₁₂ group or one N(R₁₃)SO₂R₁₄ group;
R_{7,} R₁₃, R₁₄, R₁₆, R₁₇, R₁₈, R₂₂, R₂₃, R_{26,} R₂₇, R₂₉ and R₃₀ are each independently hydrogen, C₁-C₆alkyl, C₁-C₆haloalkyl, C₃-C₇cycloalkyl, C₃-C₆alkenyl, C₃-C₆alkynyl, benzyl or phenyl;
R₈, R₂₁, R₂₅ and R₂₈ are each independently hydrogen, C₁-C₁₀ alkyl, C₁-C₈haloalkyl, C₃-C₇cycloalkyl, C₃-C₇halocycloalkyl, C₃-C₁₀alkenyl, C₃-C₈haloalkenyl, C₅-C₈cycloalkenyl, C₅-C₈halocycloalkenyl, C₃-C₈alkynyl, C₃-C₈haloalkynyl, benzyl, phenyl, furfuryl, pyridyl, thienyl, oximino, or an alkali metal, alkaline earth metal, manganese, copper, zinc, cobalt, silver, nickel, ammonium or organic ammonium cation;
R₉ is hydrogen, Si(R₁₅)₃, C(O)NR₁₆R₁₇, C(O)R₁₈, SO₂R₁₉, or C₁-C₁₀alkyl, C₃-C₁₀alkenyl, C₅-C₈cycloalkenyl, C₃-C₈cycloalkyl, C₃-C₁₀alkynyl, phenyl or benzyl, each of which may optionally be substituted with one to three halogen atoms, one hydroxyl, one benzyloxy, one C₁-C₆ alkoxy group, one phenoxy group, one OC(O)R₂₀ group, one CO2R21 group, one C(O)R₂₂ group, one C(O)R₂₃R₂₄ group or one cyano group;
R₁₀ and R₁₂ are each independently hydrogen, C₁-C₆alkyl, CO₂R₂₅, C(O)R₂₆ or C(O)NR₂₇R₂₈;
R₁₁ is hydrogen, halogen, C₁-C₆alkyl, C₁-C₆haloalkyl, C₃-C₇cycloalkyl, C₃-C₆alkenyl, C₃-C₆alkynyl, benzyl, phenyl or cyano;
R₁₉ is C₁-C₆alkyl, C₁-C₆haloalkyl, C₃-C₇cycloalkyl, C₃-C₆alkenyl, C₃-C₆alkynyl, benzyl, phenyl or cyano;
R₂₄ is hydrogen, C₁-C₆alkyl, C₁-C₆alkoxy, C₁-C₆haloalkyl, C₃-C₇cycloalkyl, C₃-C₆alkenyl, C₃-C₆alkynyl, SO₂R₂₉, C(O)R₃₀, benzyl or phenyl;
R₁₅ and R₂₀ are each independently C₁-C₆alkyl, C₃-C₆alkenyl, C₃-C₆alkynyl, benzyl or phenyl; and
X₄ is O.

7. The method according to claim 6 wherein
R is methyl or amino;
R₁ is trifluoromethyl;
R₂ is hydrogen;
X is fluorine;
R₆ is phenyl optionally substituted with any combination of one to three halogen atoms, one to three C₁-C₄alkyl groups, one to three C₁-C₄alkoxy groups, one methoxy-methyl group, one C₃-C₇cycloalkyl group, or one or two X₄R₉ groups;
R₉ C₁-C₃alkyl, C₃-C₆alkenyl, C₃-C₈cycloalkyl, C₃-C₆alkynyl, each of which may optionally be substituted with one halogen atom, one C₁-C₃ alkoxy group, one OC(O)R₂₀ group, one CO₂R₂₁ group, one C(O)R₂₂ group or one C(O)NR₂₃R₂₄ group;
R₂₀ is C₁-C₄alkyl, C₃-C₆alkenyl, C₃-C₆alkynyl, benzyl or phenyl;
R₂₁ hydrogen, C₁-C₄alkyl, C₃-C₇cycloalkyl, C₃-C₆alkenyl, C₃-C₆alkynyl, benzyl, phenyl, furfuryl, pyridyl, thienyl, oximino, or an alkali metal, alkaline earth metal, manganese, copper, zinc, cobalt, silver, nickel, ammonium or organic ammonium cation;
R₂₂ R₂₃ and R₂₄ are each independently hydrogen, C₁-C₄alkyl, C₁-C₄haloalkyl, C₃-C₇cycloalkyl, C₃-C₆alkenyl, C₃-C₆alkynyl, benzyl or phenyl; and
X₄ is O.

8. The method according to claim 7 wherein the compound is selected from the group consisting of
3-[1-(4-ethylphenyl)-5-fluoro-1H-1,2,3-benzotriazol-6-yl]-1-methyl-6-(trifluoromethyl)-2,4(1H,3H)-pyrimidinedione;
3-[5-fluoro-1-(2-methoxy-5-methylphenyl)-1H-1,2,3-benzotriazol-6-yl]-1-methyl-6-(trifluoromethyl)-2,4(1H,3H)-pyrimidinedione;
methyl {2-[5-fluoro-6-(3-methyl-2,6-dioxo-4-(trifluoromethyl)-3,6-dihydro-1(2H)-pyrimidinyl)-1H-1,2,3-benzotriazol-1-yl]-4-methylphenoxy}acetate; and
3-[1-(3-ethylphenyl)-5-fluoro-1H-1,2,3-benzotriazol-6-yl]-1-methyl-6-(trifluoromethyl)-2,4(1H,3H)-pyrimidinedione.

9. The method according to claim 5 which comprises applying the compound to the foliage of the plants or to the soil or water containing seeds or other propagating organs there at a rate of about 0.004 kg/ha to 2.0 kg/ha.

10. A herbicidal composition which comprises an inert solid or liquid carrier and a herbicidally effective amount of a compound having the formula I. 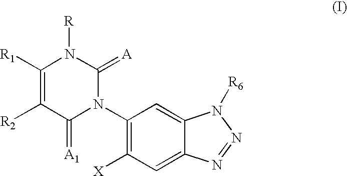
wherein R, R₁, R₂, A₆, A, A₁ and X are as defined in claim 1.

11. The composition according to claim 10 wherein
R₁ is C₁-C₃alkyl or amino;
R₁ is C₁-C₃haloalkyl;
R₂ is hydrogen;
X is halogen;
A and A₁ are O;
R₆ is phenyl optionally substituted with any combination of one to three halogen atoms, one to three C₁-C₈alkyl groups, one to three C₁-C₆alkoxy groups, one C₁-C₄alkoxy-C₁-C₄alkyl group, one C₃-C₇cycloalkyl group, one or two C₁-C₆haloalkyl groups, one C₁-C₄alkylthio group, one cyano group, one nitro group, one C(O)R₇ group, one CO₂R₈ group, one or two X₄R₉groups, one CH=CHR₁₀ group, one CH₂CH(R₁₁)R₁₂ group or one N(R₁₃) SO₂R₁₄ group;
R₇, R₁₃, R₁₄, R₁₆, R₁₇, R₁₈, R₂₂, R₂₃, R₂₆, R₂₇, R₂₉ and R₃₀ are each independently hydrogen, C₁-C₆alkyl, C₁-C₆haloalkyl, C₃-C₇cycloalkyl, C₃-C₆alkenyl, C₃-C₆alkynyl, benzyl or phenyl;
R₈, R₂₁, R₂₅ and R₂₈ are each independently hydrogen, C₁-C₁₀alkyl, C₁-C₈haloalkyl, C₃-C₇cycloalkyl, C₃-C₇halocycloalkyl, C₃-C₁₀alkenyl, C₃-C₈haloalkenyl, C₅-C₈cycloalkenyl, C₅-C₈halocycloalkenyl, C₃-C₈alkynyl, C₃-C₈haloalkynyl, benzyl, phenyl, furfuryl, pyridyl, thienyl, oximino, or an alkali metal, alkaline earth metal, manganese, copper, zinc, cobalt, silver, nickel, ammonium or organic ammonium cation;
R₉ is hydrogen, Si(R₁₅)₃, C(O)NR₁₆R₁₇, C(O)R₁₈, SO₂R₁₉, or C₁-C₁₀alkyl, C₃-C₁₀ alkenyl, C₅-C₈cycloalkenyl, C₃-C₈cycloalkyl, C₃-C₁₀alkynyl, phenyl or benzyl, each of which may optionally be substituted with one to three halogen atoms, one hydroxyl, one benzyloxy, one C₁-C₆ alkoxy group, one phenoxy group, one OC(O)R₂₀ group, one CO2R21 group, one C(O)R₂₂ group, one C(O)R₂₃R₂₄ group or one cyano group;
R₁₀ and R₁₂ are each independently hydrogen, C₁-C₆alkyl, CO₂R₂₅, C(O)R₂₆ or C(O)NR₂₇R₂₈;
R₁₁ is hydrogen, halogen, C₁-C₆alkyl, C₁-C₆haloalkyl, C₃-C₇cycloalkyl, C₃-C₆alkenyl, C₃-C₆alkynyl, benzyl, phenyl or cyano;
R₁₉ is C₁-C₆alkyl, C₁-C₆haloalkyl, C₃-C₇cycloalkyl, C₃-C₆alkenyl, C₃-C₆alkynyl, benzyl, phenyl or cyano;
R₂₄ is hydrogen, C₁-C₆alkyl, C₁-C₆alkoxy, C₁-C₆haloalkyl, C₃-C₇cycloalkyl, C₃-C₆alkenyl, C₃-C₆alkynyl, SO₂R₂₉, C(O)R₃₀, benzyl or phenyl;
R₁₅ and R20 are each independently C₁-C₆alkyl, C₃-C₆alkenyl, C₃-C₆alkynyl, benzyl or phenyl; and
X₄ is O.

12. The composition according to claim 11 wherein
R is methyl or amino;
R₁ is trifluoromethyl;
R₂ is hydrogen;
X is fluorine;
R₆ is phenyl optionally substituted with any combination of one to three halogen atoms, one to three C₁-C₄alkyl groups, one to three C₁-C₄alkoxy groups, one methoxymethyl group, one C₃-C₇cycloalkyl group, or one or two X₄R₉ groups;
R₉ C₁-C₃alkyl, C₃-C₆alkenyl, C₃-C₈cycloalkyl, C₃-C₆alkynyl, each of which may optionally be substituted with one halogen atom, one C₁-C₃ alkoxy group, one OC(O)R₂₀ group, one CO₂R₂₁ group, one C(O)R₂₂ group or one C(O)NR₂₃R₂₄ group;
R₂₀ is C₁-C₄alkyl, C₃-C₆alkenyl, C₃-C₆alkynyl, benzyl or phenyl;
R₂₁ hydrogen, C₁-C₄alkyl, C₃-C₇cycloalkyl, C₃-C₆alkenyl, C₃-C₆alkynyl, benzyl, phenyl, furfuryl, pyridyl, thienyl, oximino, or an alkali metal, alkaline earth metal, manganese, copper, zinc, cobalt, silver, nickel, ammonium or organic ammonium cation;
R₂₂ R₂₃ and R₂₄ are each independently hydrogen, C₁-C₄alkyl, C₁-C₄haloalkyl, C₃-C₇cycloalkyl, C₃-C₆alkenyl, C₃-C₆alkynyl, benzyl or phenyl; and
X₄ is O.

13. The composition according to claim 12 wherein the compound is selected from the group consisting of
3-[1-(4-ethylphenyl)-5-fluoro-1H-1,2,3-benzotriazol-6-yl]-1-methyl-6-(trifluoromethyl)-2,4(1H,3H)-pyrimidinedione;
3-[5-fluoro-1-(2-methoxy-5-methylphenyl)-1H-1,2,3-benzotriazol-6-yl]-1-methyl-6-(trifluoromethyl)-2,4(1H,3H)-pyrimidinedione;
methyl {2-[5-fluoro-6-(3-methyl-2,6-dioxo-4-(trifluoromethyl)-3,6-dihydro-1(2H)-pyrimidinyl)-1H-1,2,3-benzotriazol-1-yl]-4-methylphenoxy}acetate; and
3-[1-(3-ethylphenyl)-5-fluoro-1H-1,2,3-benzotriazol-6-yl]-1-methyl-6-(trifluoromethyl)-2,4(1H,3H)-pyrimidinedione.

14. An intermediate compound of formula VIII 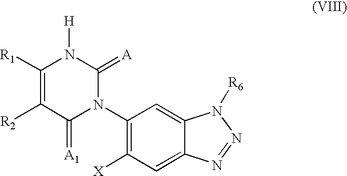
wherein R₁, R₂, R₆, A, A₁ and X are as defined in claim 1.

15. The compound according to claim 14 wherein
R₁ is C₁-C₃haloalkyl;
R₂ is hydrogen;
X is halogen;
A and A₁ are O;
R₆ is phenyl optionally substituted with any combination of one to three halogen atoms, one to three C₁-C₈alkyl groups, one to three C₁-C₆alkoxy groups, one C₁-C₄alkoxyl-C₁-C₄alkyl group, one C₃-C₇cycloalkyl group, one or two C₁-C₆haloalkyl groups, one C₁-C₄alkylthio group, one cyano group, one nitro group, one C(O)R₇ group, one CO₂R₈ group, one or two X₄R₉ groups, one CH=CHR₁₀ group, one CH₂CH(R₁₁)R₁₂ group or one N(R₁₃) SO₂R₁₄ group;
R₇, R₁₃, R₁₄, R₁₆, R₁₇, R₁₈, R₂₂, R₂₃, R₂₆, R₂₇, R₂₉ and R₃₀ are each independently hydrogen, C₁-C₆alkyl, C₁-C₆haloalkyl, C₃-C₇cycloalkyl, C₃-C₆alkenyl, C₃-C₆alkynyl, benzyl or phenyl;
R₈, R₂₁, R₂₅ and R₂₈ are each independently hydrogen, C₁-C₁alkyl, C₁-C₈haloalkyl, C₃-C₇cycloalkyl, C₃-C₇halocycloalkyl, C₃-C₁₀alkenyl, C₃-C₈haloalkenyl, C₅-C₈cycloalkenyl, C₅-C₈halocycloalkenyl, C₃-C₈alkynyl, C₃-C₈haloalkynyl, benzyl, phenyl, furfuryl, pyridyl, thienyl, oximino, or an alkali metal, alkaline earth metal, manganese, copper, zinc, cobalt, silver, nickel, ammonium or organic ammonium cation;
R₉ is hydrogen, Si(R₁₅)₃, C(O)NR₁₆R₁₇, C(O)R₁₈, SO₂R₁₉, or C₁-C₁₀alkyl, C₃-C₁₀ alkenyl, C₅-C₈cycloalkenyl, C₃-C₈cycloalkyl, C₃-C₁₀alkynyl, phenyl or benzyl, each of which may optionally be substituted with one to three halogen atoms, one hydroxyl, one benzyloxy, one C₁-C₆ alkoxy group, one phenoxy group, one OC(O)R₂₀ group, one CO2R21 group, one C(O)R₂₂ group, one C(O)R₂₃R₂₄ group or one cyano group;
R₁₀ and R₁₂ are each independently hydrogen, C₁-C₆alkyl, CO₂R₂₅, C(O)R₂₆ or C(O)NR₂₇R₂₈;
R₁₁ is hydrogen, halogen, C₁-C₆alkyl, C₁-C₆haloalkyl, C₃-C₇cycloalkyl, C₃-C₆alkenyl, C₃-C₆alkynyl, benzyl, phenyl or cyano;
R₁₉ is C₁-C₆alkyl, C₁-C₆haloalkyl, C₃-C₇cycloalkyl, C₃-C₆alkenyl, C₃-C₆alkynyl, benzyl, phenyl or cyano;
R₂₄ is hydrogen, C₁-C₆alkyl, C₁-C₆alkoxy, C₁-C₆haloalkyl, C₃-C₇cycloalkyl, C₃-C₆alkenyl, C₃-C₆alkynyl, SO₂R₂₉, C(O)R₃₀, benzyl or phenyl;
R₁₅ and R₂₀ are each independently C₁-C₆alkyl, C₃-C₆alkenyl, C₃-C₆alkynyl, benzyl or phenyl; and
X₄ is O.

16. The compound according to claim 15 wherein
R₁ is trifluoromethyl;
R₂ is hydrogen;
X is fluorine;
R₆ is phenyl optionally substituted with any combination of one to three halogen atoms, one to three C₁-C₄alkyl groups, one to three C₁-C₄alkoxy groups, one methoxymethyl group, one C₃-C₇cycloalkyl group, or one or two X₄R₉, groups;
R₉ C₁-C₃alkyl, C₃-C₆alkenyl, C₃-C₈cycloalkyl, C₃-C₆alkynyl, each of which may optionally be substituted with one halogen atom, one C₁-C₃ alkoxy group, one OC(O)R₂₀ group, one CO₂R₂₁ group, one C(O)R₂₂ group or one C(O)NR₂₃R₂₄ group;
R₂₀ is C₁-C₄alkyl, C₃-C₆alkenyl, C₃-C₆alkynyl, benzyl or phenyl;
R₂₁ hydrogen, C₁-C₄alkyl, C₃-C₇cycloalkyl, C₃-C₆alkenyl, C₃-C₆alkynyl, benzyl, phenyl, furfuryl, pyridyl, thienyl, oximino, or an alkali metal, alkaline earth metal, manganese, copper, zinc, cobalt, silver, nickel, ammonium or organic ammonium cation;
R₂₂ R₂₃ and R₂₄ are each independently hydrogen, C₁-C₄alkyl, C₁-C₄haloalkyl, C₃-C₇cycloalkyl, C₃-C₆alkenyl, C₃-C₆alkynyl, benzyl or phenyl; and
X₄ is O.

17. The compound according to claim 16 wherein the compound is selected from the group consisting of
3-[1-(4-ethylphenyl)-5-fluoro-1H-1,2,3-benzotriazol-6-yl]-6-(trifluoromethyl)-2,4(1H,3H)-pyrimidinedione;
3-[5-fluoro-1-(2-methoxy-5-methylphenyl)-1H-1,2,3-benzotriazol-6-yl]-6-(trifluoromethyl)-2,4(1H,3H)-pyrimidinedione; and
3-[1-(3-ethylphenyl)-5-fluoro-1H-1,2,3-benzotriazol-6-yl]-6-(trifluoromethyl)-2,4(1H,3H)-pyrimidinedione.
